(19) United States
(12) Patent Application Publication
(10) Pub. No.: US 2021/0008253 A1
(43) Pub. Date: Jan. 14, 2021

(54) ELONGATE SCAFFOLD COMPRISING INNER AND OUTER PORTION

(71) Applicant: The Electrospinning Company Limited, Didcot (GB)

(72) Inventors: Robert James McKean, Didcot (GB); Brendan John Robb, Didcot (GB); Teodor-Matei Cirstea, Didcot (GB)

(21) Appl. No.: 15/733,560

(22) PCT Filed: Jan. 29, 2019
(86) PCT No.: PCT/GB2019/050236

§ 371 Date: Aug. 27, 2020

(30) Foreign Application Priority Data
Mar. 2, 2018 (GB) 1803435.5

(51) Int. Cl. A61L 27/56; A61L 27/18; C08L 67/04; A61L 27/38; A61L 27/54; A61L 27/58; D01D 5/00

(52) U.S. Cl. CPC A61L 27/56; A61L 2430/38; C08L 67/04; A61L 27/3834; A61L 27/54; A61L 27/58; D01D 5/003; D01D 5/0076; A61L 2430/02; A61L 2430/06; A61L 2430/10; A61L 2430/16; A61L 2430/20; A61L 2430/22; A61L 2430/26; A61L 2430/28; A61L 2430/30; A61L 27/18

(57) ABSTRACT

The invention relates to an elongate scaffold comprising: an inner portion comprising a polymer; and an outer portion comprising a porous, nonwoven network of polymer fibers, wherein the packing density of the inner portion is greater than the packing density of the outer portion; wherein the inner portion (a) comprises a plurality of polymer fibers twisted around one another or (b) is a solid core comprising the polymer. The invention also relates to a scaffold precursor and a process for producing a scaffold, comprising twisting a scaffold precursor of the invention along its length. Further provided is a hybrid composition comprising the scaffold and cells and/or an active agent such as a drug, a nucleic acid, a nucleotide, a protein, a polypeptide, or an exosome. Therapeutic methods and uses of such hybrid compositions are also provided, for instance in tissue repair, wound healing, and in the treatment of a cardiac, bone, cartilage, tendon, ligament, liver, kidney joint, spleen, eye, spinal disc, connective tissue, or lung injury or disease or cancer, or an infection in a patient, and as tissue fillers for reconstructive or cosmetic applications.

### CROSS-REFERENCE TO RELATED APPLICATIONS

This application is a 371 National Stage Entry of International Patent Application No. PCT/GB2019/050236 filed on Jan. 29, 2019, which claims the benefit of British Patent Application No. 1803435.5 filed on Mar. 2, 2018, the contents of which are incorporated herein by reference in their entirety.

### FIELD OF THE INVENTION

The invention relates to a scaffold suitable for supporting cell growth, and which is suitable for use as a delivery vehicle, for delivering cells or other active ingredients to a particular target tissue in vivo. The invention also relates to a process for producing the scaffold, and to a scaffold precursor that may be used in the process. Hybrid compositions, comprising the scaffold and cells or other active ingredients, are also provided, as are therapeutic methods and uses involving the hybrid compositions.

### BACKGROUND OF THE INVENTION

A variety of different cell types and active agents can be used in repairing damaged or diseased tissue, including therapeutic cells. Once administered to the patient, therapeutic cells can exert their effects through paracrine signaling and by promoting survival, repair, and regeneration of the neighboring cells in the damaged or diseased tissue. However, the repair of damaged or diseased tissue by therapeutic cells, or indeed by biomolecules and other active agents, is often inefficient because (1) the cells, biomolecule or active agent can only access and repair the surface of the damaged or diseased tissue, but cannot reach deeper parts of the damaged or diseased tissue, (2) they only remain in the optimal location for repair for a limited time period and (3) the local microenvironment in which they are placed is not optimal to promote repair and regeneration. These limitations often result in a suboptimal or partial repair of the damaged or diseased tissue.

US 2016-0228608 A1 discloses a scaffold for tissue repair or wound dressing comprising: a material layer; a polymer fiber layer; and an adhesive component between the material layer and the polymer fiber layer. The scaffold may comprise cells. The adhesive layer may be produced by electrospinning.

Tan, Zhikai, et al. “Electrospun vein grafts with high cell infiltration for vascular tissue engineering”, Materials Science & Engineering. C.81, 2017, 407-415 discloses bilayered scaffolds consisting of a highly porous external layer and a relatively dense internal layer with longitudinally aligned fibers. Electrospinning is used to deposit the fibers of the external layer over the internal layer. Tubular grafts were cut longitudinally into mats and seeded with cells.

US 2004-0054406 A1 discloses a vascular prosthesis comprising a first layer having a predetermined first porosity and a second layer having a predetermined second porosity, wherein the first layer and the second layer are each made of first and second electrospun polymer fibers. The vascular prosthesis may include a coiled pattern, for instance, formed from a wound filament.

### SUMMARY OF THE INVENTION

The present invention provides a scaffold which is suitable for use as a delivery vehicle, for delivering cells, biomolecules and active agents, and in particular therapeutic cells, to a particular target tissue in vivo. The scaffold comprises a porous network of fibers. The porous nature of this network allows cells to infiltrate into, and proliferate through, the network, such that cell cultures can grow on the surface of, or indeed within, the porous network and can adhere to the network efficiently. Furthermore, the microenvironment provided by such a porous network of fibers facilitates and supports the growth of three-dimensional (3D) cell populations for a wide variety of cell types, including cell lines, stem cells and primary cells. This property of such a porous network of fibers when supported in a multiwell plate has been demonstrated experimentally in WO/2013/117926 A1. Advantageously, cells in 3D populations have 100% of their cell surface area exposed to other cells and matrix, which stimulates specific signaling pathways to initiate tissue-specific gene expression and stimulate the production of extracellular matrix (ECM). As a result, cultures grown in such a porous network of fibers are similar to cells found within tissues in the body and more accurately resemble the in vivo environment.

If a porous network of fibers is unsupported it can stretch or deform under mechanical stress, causing adverse changes to the porosity and pore size in the network that facilitates cell growth. The stresses to which such a porous network may be subjected, e.g. during or following administration to a patient, can therefore have a detrimental effect on the ability of the network to function as a scaffold that facilitates growth of three-dimensional (3D) cell populations, for a wide variety of cell types.

Accordingly, the scaffold of the invention advantageously further comprises a component that is suitable for providing mechanical strength and for maintaining the integrity of the open pore structure of the porous network of fibers. This second component can advantageously restrict deformation or stretching of the porous network of fibers, and thereby minimize consequential adverse changes to the porosity and pore size of the porous network that facilitates cell growth.

The scaffold of the invention may be elongate, or cylinder-shaped. Whilst fibrous yarns, which are well known to those skilled in the art (O'Connor and McGuinness, Proc Inst Mech Eng H. 2016 November; 230(11):987-998. doi: 10.1177/0954411916656664), provide structures potentially suitable for implantation or injection via a needle or catheter, the porosity and/or small pore size resulting from the twisting of the fibers into a yarn limits the ability for cellular ingrowth into the volume of the yarn. The present invention, on the other hand, enables the scaffold porosity and pore size in a nonwoven component to be controlled and maintained, which are important factors that affect whether or not cell populations can successfully grow into the scaffold. As the skilled person will appreciate, a scaffold should be sufficiently porous, with large enough pores, to allow cells to infiltrate the scaffold and grow in the 3D microenvironment. The scaffold of the invention therefore facilitates the formation of a hybrid composition, as described hereinafter, which contains a high cell density per unit volume of the scaffold, relative to a solid cylinder or a woven, yarn-like structure.

Such hybrid compositions can be used to deliver therapeutic cells into a target tissue, for instance into a damaged or diseased tissue, more effectively. For instance, such a hybrid composition is capable of providing the cells with access to the optimal location to effect optimal repair of damaged or diseased tissue. The cells may, for example, reach deeper parts of the damaged or diseased tissue. The hybrid composition is also capable of retaining the cells at the optimal location to effect optimal repair of damaged or diseased tissue. The hybrid composition also creates its own optimal microenvironment for optimal repair and regeneration.

Accordingly, in a broad aspect, the invention provides a scaffold comprising a first portion which is suitable for supporting cell growth and which comprises a porous network of fibers, and a second portion which is suitable for maintaining the integrity of the porous network of fibers. The fibers in the first portion are typically polymeric. The network of fibers of the first portion is generally a nonwoven network, i.e. the fiber is typically randomly orientated in the porous network. The second portion also typically comprises a polymer.

As discussed above the scaffold is typically elongate, and can for example be cylinder-shaped, as this can facilitate delivery to tissue by injection or catheter. However, as will be discussed further below, the scaffold may in principle be any shape, and other specific shapes are also envisaged.

The first portion of the scaffold, which comprises the porous network of fibers, is typically an outer portion, and the second portion of the scaffold, which is suitable for maintaining the integrity of the porous network of fibers, is usually an inner portion. Then, when a hybrid composition comprising the scaffold is delivered in vivo, the cells, biomolecules or active agents being delivered (which are generally within the scaffold, on the scaffold, or both) have maximum contact with the surrounding tissue.

However, other embodiments of the scaffold are also envisaged in which the first portion of the scaffold, comprising the porous network of fibers, is within the second portion of the scaffold. Thus, the first portion may be an inner portion and the second portion may be an outer portion. In one such embodiment, the inner portion comprising the porous, nonwoven network of polymer fibers is disposed within a protective outer portion. In this embodiment, the outer portion may protect the integrity of a cell culture situated within, on, and/or around a porous inner portion, and prevent the cells from being sloughed off the inner portion. Thus, the invention also provides a hybrid composition comprising a scaffold of this embodiment and cells, wherein the cells are situated in and/or on, the inner first portion, and contained within the outer second portion. The cells may be adherent therapeutic cells.

The second portion of the scaffold, which is suitable for maintaining the integrity of the porous network of fibers in the first portion, generally has a packing density which is greater than the packing density of the first portion, especially in embodiments where the second portion is an inner portion and the first portion is an outer portion. For instance, while the first portion comprises a porous network of fibers, making the packing density of the first portion relatively low, the second portion is generally more compacted, making the packing density of the second portion relatively higher. For instance, the second portion may comprise a plurality of polymer fibers that are aligned and/or twisted around one another to form a relatively compact yarn-like structure, wherein the packing density of the second portion is greater than the packing density of the first portion, or the second portion may not comprise fibers but a solid polymer. The solid polymer—which may be described as a solid polymer core if the second portion is an inner portion—may have relatively little or no porosity, i.e. it may be nonporous or have a lower porosity than the porous network of fibers.

Thus, in one preferred aspect, the invention provides a scaffold comprising: an inner portion comprising a polymer; and an outer portion comprising a porous, nonwoven network of polymer fibers, wherein the packing density of the inner portion is greater than the packing density of the outer portion. Although any shape of scaffold may be employed, as discussed above, the scaffold in this preferred aspect is often an elongate scaffold.

Thus, the invention provides an elongate scaffold comprising: an inner portion comprising a polymer; and an outer portion comprising a porous, nonwoven network of polymer fibers, wherein the packing density of the inner portion is greater than the packing density of the outer portion.

The invention also provides a scaffold precursor which is an elongate strip comprising a plurality of layers of polymer fibers, the plurality of layers comprising: a first region, comprising at least one first layer of nonwoven polymer fibers; and a second region, disposed on the first region, the second region comprising at least one layer comprising aligned polymer fibers which are orientated along the length of the strip. The scaffold precursor of the invention may optionally further comprise a third region, which is disposed on the second region, which third region comprises at least one further layer of nonwoven polymer fibers.

The invention further provides a process for producing a scaffold, which process comprises twisting a scaffold precursor of the invention along its length.

The invention also provides a scaffold which is obtainable by a process of the invention for producing a scaffold. A scaffold obtained by a process of the invention for producing a scaffold, is also provided.

The invention further provides a hybrid composition comprising: (i) cells, a biomolecule or other active agent; and (ii) a scaffold of the invention, a scaffold precursor of the invention, or a scaffold which is obtainable by or obtained by a process of the invention for producing a scaffold. The biomolecule or other active agent may be a drug, a nucleic acid, a nucleotide, a protein, a polypeptide, or an exosome. The nucleic acid may comprise DNA, RNA, RNAi, SaRNA or SiRNA. Optionally, the hybrid composition comprises (i) cells, for instance adherent therapeutic cells, and (ii) a scaffold of the invention, a scaffold precursor of the invention, or a scaffold which is obtainable by or obtained by a process of the invention for producing a scaffold. The cells may be disposed within the porous network of fibers in the scaffold or scaffold precursor. The cells may be disposed in pores of the scaffold or scaffold precursor. The cells may be disposed on (e.g. may adhere to) the surface of the scaffold or scaffold precursor. The cells may be disposed in pores of the scaffold or scaffold precursor and may also be disposed on (e.g. may adhere to) the surface of the scaffold or scaffold precursor. Preferably, the cells are in the outer portion of the scaffold or scaffold precursor.

The invention further provides a hybrid composition comprising: (i) adherent therapeutic cells, a drug, a nucleic acid, a nucleotide, a protein, a polypeptide or an exosome, optionally wherein the nucleic acid comprises DNA, RNA, RNAi, SaRNA or SiRNA; and (ii) a scaffold of the invention. Optionally, the hybrid composition comprises adherent therapeutic cells and said scaffold, and the adherent therapeutic cells adhere to the outer surface of the scaffold, or are disposed in pores of the scaffold, or both.

The invention additionally provides a process for producing a hybrid composition of the invention as defined above, comprising combining (i) a scaffold of the invention, and (ii) cells, a biomolecule or other active agent, in a culture vessel.

In a preferred aspect, the invention provides a process for producing a hybrid composition of the invention as defined above, comprising combining (i) a scaffold of the invention, and (ii) adherent therapeutic cells, a drug, a nucleic acid, a nucleotide, a protein, a polypeptide or an exosome in a culture vessel.

The invention further provides a hybrid composition of the invention as defined above, comprising combining (i) a scaffold of the invention, and (ii) adherent therapeutic cells, a drug, a nucleic acid, a nucleotide, a protein, a polypeptide or an exosome in an insertion device, for example a syringe or needle prefilled with the scaffolds such that the hybrid composition is formed in situ prior to administration.

The invention further provides a hybrid composition of the invention for use in a method for treatment of the human or animal body by therapy.

Further provided is a method of repairing a damaged or diseased tissue in a patient, comprising contacting the damaged or diseased tissue with one or more hybrid compositions of the invention, and thereby treating the damaged or diseased tissue in the patient. The hybrid composition may comprise adherent therapeutic cells and said scaffold. The composition may comprise a therapeutically effective number of the adherent therapeutic cells.

The invention additionally provides a hybrid composition of the invention for use in a method of treating a cardiac, bone, cartilage, tendon, ligament, liver, kidney, joint, spleen, eye, spinal disc, connective tissue, or lung injury or disease in a patient, for use in treating an infection in a patient, for use in treating cancer in a patient, for use in wound healing, or for tissue fillers for reconstructive or cosmetic applications.

### BRIEF DESCRIPTION OF THE FIGURES

FIGS. 1A and 1B show SEM images of: (A) an outer, nonwoven layer of the trilayer sheet produced by electrospinning during the manufacture of the scaffold precursor in accordance with Example 1; and (B) one end of a PLGA cylinder-shaped scaffold that has been produced by twisting a trilayer scaffold precursor strip in accordance with Example 1.

FIG. 2 shows an optical microscope composite image of three PLGA cylinder-shaped scaffolds in accordance with Example 1, exemplifying different numbers of twists per mm.

FIG. 3 shows iMP cells attached to a cylinder-shaped scaffold and stained with (A) Phalloidin (left) and (B) Hoechst (right) as described in Example 2 herein.

FIG. 4 shows (A) the injection of a hybrid composition of the invention into a sheep's heart (left) as described in Example 3 herein, and (B) that the cylinder-shaped scaffold has left the syringe and needle and entered the myocardium.

### DETAILED DESCRIPTION OF THE INVENTION

The invention relates to a scaffold comprising a first portion which is suitable for supporting cell growth and which comprises a porous network of fibers, and a second portion which is suitable for maintaining the integrity of the porous network of fibers. The porous network of fibers of the first portion is generally a nonwoven network, i.e. the fiber is typically randomly orientated in the porous network. The fibers in the first portion are typically polymeric. The second portion also typically comprises a polymer, in which case the polymer of the second portion may be the same polymer as, or a different polymer from, the polymer in the first portion. Often, it is the same. Suitable polymers are discussed in more detail further below but include, for example, biocompatible and bioabsorbable polymers such as poly(lactide), poly(glycolide), poly(lactide-co-glycolide) (PLGA) and polycaprolactone (PCL), to name but a few. The scaffold is suitable for use as a delivery vehicle, for delivering cells, biomolecules and active agents, and in particular therapeutic cells, to a particular target tissue in vivo.

Usually the scaffold is elongate and can for example be cylinder-shaped. However, any shape may in principle be employed. For instance, the shape may be a tetrahedron, a square pyramid, a hexagonal pyramid, a cube, a cuboid, a triangular prism, an octahedron, a pentagonal prism, a hexagonal prism, a dodecahedron, a sphere, an ellipsoid, an icosahedron, a cone, a ring (or doughnut) or a cylinder. The shape may be a patch. The shape may be any size. The largest dimension of the shape may advantageously be approximately the same length as the depth of the damage or disease in the tissue which is to be treated using the composition. The largest dimension of the shape is preferably designed such that the composition can penetrate a damaged or diseased tissue to a prescribed depth. Suitable lengths of the largest dimension include, but are not limited to, at least about 200 μm in length, at least about 500 μm in length, at least about 1 mm in length, at least about 2 mm in length, at least about 3 mm on length, at least about 4 mm in length, at least about 5 mm in length, at least about 10 mm in length, at least about 15 mm in length, at least about 20 mm in length, at least about 100 mm in length, at least about 200 mm in length, or at least about 300 mm in length. The length of the largest dimension of the shape is typically determined by its intended use, and/or its ability to be manipulated, e.g. before, during or after cell growth on or within the scaffold, or before or during administration of a cell-laden scaffold to a patient.

However, other embodiments of the scaffold are also envisaged in which the length of the scaffold, or scaffold precursor, is significantly longer, such as, but not limited to, at least about 1000 mm, at least about 2000 mm, at least about 3000 mm, or at least about 4000 mm. Such embodiments, when combined with cells, biomolecules and active agents, and in particular therapeutic cells, to provide the hybrid composition of the invention, are intended to be utilized as therapeutically active sutures.

The first portion of the scaffold, which comprises the porous network of fibers, is typically an outer portion, and the second portion of the scaffold, which is suitable for maintaining the integrity of the porous network of fibers, is usually an inner portion. Then, when a hybrid composition comprising the scaffold is delivered in vivo, the cells, biomolecules or active agents being delivered (which are generally within the outer portion, on the surface of the outer portion, or both) have maximum contact with the surrounding tissue.

The inner portion generally comprises a polymer which acts as a structural support to the scaffold. Preferably, the inner portion is continuous, and is completely surrounded by the outer portion. Preferably, the scaffold does not contain a hollow void (for instance, at its center). Accordingly, in preferred embodiments, the scaffold is not the shape of a hollow tube.

However, other embodiments of the scaffold are also envisaged in which the first portion of the scaffold, comprising the porous network of fibers, is within the second portion of the scaffold. Thus, the first portion may be an inner portion and the second portion may be an outer portion.

Accordingly, in one embodiment, the inner portion comprising the porous, nonwoven network of polymer fibers is disposed within a protective outer portion. Further features of this embodiment may be as defined hereinbelow. In this embodiment, aligned polymer fibers may be employed on the outside to protect the integrity of a cell culture situated within, on, and/or around a porous inner portion, and prevent the cells from being sloughed off the inner portion. Thus, the invention also provides a hybrid composition comprising a scaffold of this embodiment and cells, wherein the cells are situated in and/or on the inner first portion but are also contained within the outer second portion. The cells may be adherent therapeutic cells.

Usually, however, the first portion (comprising the porous network of fibers) is an outer portion, and the second portion is an inner portion.

The second portion of the scaffold, which is suitable for maintaining the integrity of the porous network of fibers in the first portion, generally has a packing density which is greater than the packing density of the first portion.

The term “packing density”, as used herein with respect to a particular portion of the scaffold (i.e. with respect to one of the first and second portions, or with respect to one of the inner and outer portions) means the fraction of the volume of space which that portion occupies which is filled by the material (e.g. polymer) of which that portion consists.

For instance, while the first portion comprises a porous network of fibers, making the packing density of the first portion relatively low, the second portion is generally more compacted, making the packing density of the second portion relatively high. For instance, the second portion may comprise a plurality of polymer fibers which are aligned to form a relatively compact structure. These may be annealed or twisted around one another to form the compact structure or may be closely packed or interwoven to form a compact structure. The twisting is generally along the length of the polymer fibers and is distinct from the coiled structures (filaments) disclosed in US 2004-0054406 A1. They are typically twisted or annealed around one another to form a compact, yarn-like structure. Alternatively, instead of comprising a plurality of aligned fibers, the second portion may comprise a solid polymer structure—which may be described as a solid polymer core if the second portion is an inner portion—which is larger than a single fiber. By “larger”, it may for instance be an elongate structure, e.g. in the shape of a wire, with a diameter which is at least 10 times, for instance at least 50 times, or at least 100 times, the average diameter of the fibers in the first portion (comprising the porous network of fibers). The solid polymer may have relatively little or no porosity, i.e. it may be nonporous or have a much lower porosity than the porous network of fibers. In each of these cases, the packing density of the second portion is greater than that of the first portion.

Thus, in one preferred aspect, the invention provides a scaffold comprising: an inner portion comprising a polymer; and an outer portion comprising a porous, nonwoven network of polymer fibers, wherein the packing density of the inner portion is greater than the packing density of the outer portion. Although any shape of scaffold may be employed, as discussed above, the scaffold in this preferred aspect is often an elongate scaffold.

Thus, a preferred aspect of the invention provides an elongate scaffold comprising: an inner portion comprising a polymer; and an outer portion comprising a porous, nonwoven network of polymer fibers, wherein the packing density of the inner portion is greater than the packing density of the outer portion. Further features of this embodiment may be as defined hereinbelow.

The polymer of the inner portion may be the same polymer as, or a different polymer from, the polymer of the fibers in the inner portion. Often, it is the same polymer. Suitable polymers are discussed further hereinbelow.

The inner portion typically comprises a plurality of polymer fibers twisted around one another, or a solid core comprising the polymer. When the inner portion comprises a solid core comprising the polymer, the solid polymer core is typically an elongate structure, e.g. in the shape of a wire; it typically has a diameter which is at least 10 times, for instance at least 50 times, or at least 100 times, the average (mean) diameter of the fibers in the outer portion of the scaffold. The solid core may have relatively little or no porosity, i.e. it may be nonporous, or it may have a porosity which is less than the porosity of the outer portion comprising the porous network of fibers.

Usually, however, the inner portion comprises a plurality of polymer fibers twisted and/or annealed around one another into a larger and stronger form. The twisting is generally along the length of the polymer fibers.

The inner portion typically comprises at least 10 polymer fibers twisted around one another, and more typically at least 25 polymer fibers twisted around one another. The inner portion may for instance comprise at least 50 polymer fibers twisted around one another, at least 75 polymer fibers twisted around one another, or for example at least 100 polymer fibers twisted around one another.

Typically, the inner portion comprises said plurality of polymer fibers twisted around one another wherein the plurality of polymer fibers has an average number of twists per mm of length of the inner portion, of from 0.1 to 4. The plurality of polymer fibers may for example have an average number of twists per mm of length of the inner portion, of from 0.3 to 3, for instance from 0.5 to 2.0, for example about 1. The plurality of polymer fibers may for example have an average number of twists per mm of length of the inner portion, of 1.0.

A way in which the polymer fibers of the inner portion may be twisted around one another is described further hereinbelow in the detailed description of the process of the invention. Thus, a scaffold precursor, comprising a layer of aligned polymer fibers and at least one layer of nonwoven polymer fibers, is twisted along its length such that the aligned polymer fibers become twisted around one another.

Typically, therefore, the inner portion comprises said plurality of polymer fibers twisted around one another, which is a plurality of aligned fibers twisted around one another.

Often, the inner portion runs the length of the scaffold. The inner portion may be parallel to the length of the scaffold.

In the scaffold of the invention, the outer portion is disposed around at least part of the inner portion. Due to the nature of the process of the invention by which an elongate scaffold of the invention is typically formed (i.e. by twisting a scaffold precursor, which comprises at least one layer of nonwoven fiber and a layer of aligned fiber, along its length) sections along the length of the inner portion (i.e. the portion formed from the aligned fiber in the scaffold precursor) may still be exposed after the twisting. Thus, in the scaffold of the invention, the outer portion may or may not be disposed around the inner portion along the whole length of the scaffold (or inner portion of the scaffold) but only along part of its length. Indeed, in some embodiments, the outer portion is disposed around the inner portion only along part of the length of the scaffold, whereas in other embodiments, the outer portion is disposed around the inner portion along the length of the scaffold (i.e. along the whole length of the inner portion of the scaffold). Thus, in the scaffold of the invention, the outer portion is disposed around at least part of the inner portion along the length of the inner portion.

Typically, the scaffold of the invention is cylinder-shaped. The scaffold is typically approximately straight. Alternatively, the scaffold may be curved. The scaffold may have any number of curves or bends. The scaffold may form a corkscrew shape. This may facilitate the retention of the device in the target tissue.

Usually, the length of the scaffold is at least about 2 times the diameter of the scaffold. The length of the scaffold may for instance be at least about 5 times the diameter of the scaffold, or for instance at least about 10 times the diameter of the scaffold. It may for example be at least about 50 times the diameter of the scaffold, or for example at least about 100 times the diameter of the scaffold, for instance at least about 500 times the diameter of the scaffold, or for instance about 1000 times the diameter of the scaffold, or at least about 10,000 times the diameter of the scaffold. The length may be up to about 20,000 times the diameter of the scaffold.

Often, the length of the scaffold is from about 2 to about 1000 times the diameter of the scaffold, and is more typically from about 5 to about 1000 times the diameter of the scaffold, for instance from about 8 to about 1000 times the diameter of the scaffold. It may for example be from about 8 to about 800 times the diameter of the scaffold, e.g. from about 10 to about 500 times the diameter of the scaffold, or from about 10 to about 300 times the diameter of the scaffold. The length of the scaffold may for instance be from about 10 to about 50 times the diameter of the scaffold, or from about 10 to about 30 times the diameter of the scaffold.

The scaffold of the invention may have a diameter of from about 100 μm to about 1000 μm, for instance from about 150 μm to about 750 μm, or for example from about 200 μm to about 600 μm. The scaffold may for instance have a diameter of from about 200 μm to about 500 μm, or for example from about 300 μm to about 400 μm.

The scaffold of the invention may have a length of at least 1 mm, for instance at least 2 mm. The scaffold may for instance have a length of at least 4 mm, or for example at least 5 mm. The scaffold of the invention may for instance have a length of at least 10 mm.

The scaffold of the invention may have a length of from about 1 mm to about 300 mm, for instance from about 2 mm to about 100 mm. The scaffold may for instance have a length of from about 4 mm to about 80 mm, or for example from about 5 mm to about 60 mm. The scaffold of the invention may for instance have a length of from about 1 mm to about 10 mm, for instance from about 2 mm to about 10 mm, or, for instance, from about 4 mm to about 8 mm, for example from about 4 mm to about 6 mm.

The polymer fibers in the porous, nonwoven network of fibers in the outer portion do not have any particular orientation to speak of, i.e. the fiber in the porous, nonwoven network is typically randomly-oriented or at least approaching random orientation. The degree of alignment of the polymer fibers in the outer portion is therefore low, if not entirely random.

Where polymer fibers are present in the inner portion, e.g. when the inner portion comprises a plurality of polymer fibers twisted around one another, the degree of alignment of the polymer fibers in the inner portion is greater than or equal to that in the outer portion. Indeed, the inner portion may comprise a plurality of aligned polymer fibers twisted around one another. Prior to twisting, the aligned polymer fibers may have been aligned substantially parallel to one another.

In the scaffold of the invention, the mean diameter of the polymer fibers is usually from about 500 nm to about 10 μm. Typically, the mean diameter of the polymer fibers is from about 1 μm to about 5 μm, for instance from about 2 μm to about 5 μm. The mean diameter of the polymer fibers may for example from about 2 μm to about 4 μm, for example about 3 μm. The relative standard deviation from the mean is typically less than or equal to 25%.

Thus, the mean diameter of the polymer fibers in the outer portion of the scaffold of the invention, e.g. when the outer portion comprises said porous, nonwoven network of polymer fibers, is usually from about 500 nm to about 10 μm. Typically, the mean diameter of the polymer fibers in the outer portion is from about 1 μm to about 5 μm, for instance from about 2 μm to about 5 μm. The mean diameter of the polymer fibers in the outer portion may for example from about 2 μm to about 4 μm, for example about 3 μm. The relative standard deviation from the mean is typically less than or equal to 25%.

Similarly, where fibers are present in the inner portion of the scaffold, e.g. when the inner portion comprises a plurality of polymer fibers annealed together and/or twisted around one another, the mean diameter of the polymer fibers in the inner portion of the scaffold of the invention is usually from about 500 nm to about 10 μm. Typically, the mean diameter of the polymer fibers in the inner portion is from about 1 μm to about 5 μm, for instance from about 2 μm to about 5 μm. The mean diameter of the polymer fibers in the inner portion may for example from about 2 μm to about 4 μm, for example about 3 μm. The relative standard deviation from the mean is typically less than or equal to 25%.

Often, the mean diameter of the polymer fibers in the outer portion of the scaffold of the invention is the same as the mean diameter of the polymer fibers in the inner portion of the scaffold.

The porous network of polymer fibers in the outer portion typically has a mean pore size which is greater than the mean pore size of the inner portion. The porous network of polymer fibers in the outer portion typically also has a porosity which is greater than the porosity of the inner portion.

For instance, the porous, nonwoven network of polymer fibers in the outer portion of the scaffold of the invention typically has a porosity which is equal to or greater than 50%. The porous network of polymer fibers in the outer portion may have a mean pore size of, for example, from 10 μm to 100 μm. Pore size can be difficult to measure accurately, though, as pore size depends on how far through the scaffold you measure, and no two pores are the same shape due to the random orientation of the nanofibers. The pore size is tuned roughly to match a typical cell diameter (approx. 20 microns) however the loose nature of the fibers in the nonwoven portion allows for cells to migrate into the scaffold by pushing the nanofibers aside. Such porosity is beneficial in terms of allowing cells to proliferate through the outer portion of the scaffold.

The polymer fibers in the inner and outer portions can be produced by electrospinning, as detailed further below, or by other suitable methods which are known to the skilled person including, but not limited to, melt spinning, dry spinning, wet spinning and extrusion. Electrospinning is preferred.

Thus, the polymer fibers in the porous network of the outer portion may comprise electrospun, melt-spun, dry-spun, wet-spun, or extruded fibers. Usually, however, the polymer fibers in the porous network of the outer portion are electrospun fibers. Similarly, where fibers are present in the inner portion of the scaffold, e.g. when the inner portion comprises a plurality of polymer fibers twisted around one another, the fibers of the inner portion may comprise electrospun, melt-spun, dry-spun, wet-spun or extruded fibers. Usually, however, the polymer fibers of the inner portion are electrospun fibers.

The polymer fibers of the inner portion may comprise the same polymer as or a different polymer from the polymer fibers employed in the outer portion. Typically, however, the same polymer is employed for the fibers of the inner and outer portions.

The fibers of the inner and outer portions typically comprise, or for instance consist of, a polymer which is both bioabsorbable and biocompatible, for instance poly(lactide), poly(glycolide), poly(lactide-co-glycolide) (PLGA) or polycaprolactone (PCL). Polyhydroxybutyrate or a poly(ester urethane) may alternatively be employed.

More generally, the fibers of the inner and outer portions may comprise a polymer which is the same or different and is independently selected from the following:

poly(lactide); poly(glycolide); poly(lactide-co-glycolide) (PLGA); polycaprolactone (PCL); polyhydroxybutyrate; poly(ε-caprolactone); polystyrene; polyethylene; polypropylene; poly(ethylene oxide); a poly(ester urethane); poly(vinyl alcohol); polyacrylonitrile; polylactide; polyglycolide; polyurethane; polycarbonate; polyimide; polyamide; aliphatic polyamide; aromatic polyamide; polybenzimidazole; poly(ethylene terephthalate); poly[ethylene-co-(vinyl acetate)]; poly(vinyl chloride); poly(methyl methacrylate); poly(vinyl butyral); poly(vinylidene fluoride); poly(vinylidene fluoride-co-hexafluoropropylene); cellulose acetate; poly(vinyl acetate); poly(acrylic acid); poly(methacrylic acid); polyacrylamide; polyvinylpyrrolidone; poly(phenylene sulfide); hydroxypropylcellulose; polyvinylidene chloride, polytetrafluoroethylene, a polyacrylate, a polymethacrylate, a polyester, a polysulfone, a polyolefin, polysilsesquioxane, silicone, epoxy, cyanate ester, a bis-maleimide polymer; polyketone, polyether, polyamine, polyphosphazene, polysulfide, an organic/inorganic hybrid polymer or a copolymer thereof, for instance, poly(lactide-co-glycolide); polylactide-co-poly(ε-caprolactone) or poly(L-lactide)-co-poly(ε-caprolactone); or a blend thereof, for instance a blend of poly(vinyl alcohol) and poly(acrylic acid).

The fibers of the inner and outer portions may independently comprise, or consist of, a bioerodible polymer, for instance a bioerodible polymer selected from poly(lactide), poly(glycolide), poly(lactide-co-glycolide) (PLGA), polycaprolactone (PCL), poly(ε-caprolactone) (PCL), polyhydroxybutyrate and poly(ester urethanes).

The fibers of the inner and outer portions may alternatively for instance independently comprise, or consist of, a biopolymer, or a blend of a biopolymer with a synthetic polymer. The following biopolymers and blends of biopolymers with synthetic polymers may for instance be used:

collagen; collagen/poly(ethylene oxide); collagen/poly(ε-caprolactone); collagen/polylactide-co-poly(ε-caprolactone); gelatin; gelatin/poly(ε-caprolactone); gelatin/poly(ethylene oxide); casein/poly(vinyl alcohol); casein/poly(ethylene oxide); lipase; cellulase/poly(vinyl alcohol); bovine serum albumin/poly(vinyl alcohol); luciferase/poly(vinyl alcohol); α-chymotrypsin; fibrinogen; silk; regenerated silk; regenerated Bombyx mori silk; Bombyx mori silk/poly(ethylene oxide); silk fibroin; silk fibroin/chitosan; silk fibroin/chitin; silk/poly(ethylene oxide) (coaxial); artificial spider silk; chitin; chitosan; chitosan/poly(ethylene oxide); chitosan/poly(vinyl alcohol); quaternized chitosan/poly(vinyl alcohol); hexanoylchitosan/polylactide; cellulose; or cellulose acetate.

The fibers of the inner and outer portions may for instance comprise, or consist of, a blend of two or more polymers, a copolymer (which may for instance be a block copolymer), or a blend of a polymer with an inorganic material.

Nonlimiting examples of such blends blend of two or more polymers include a polyvinylpyrrolidone/polylactide blend; a polyaniline/polystyrene blend; a polyaniline/poly(ethylene oxide) blend; a poly(vinyl chloride)/polyurethane blend, a poly[(m-phenylene vinylene)-co-(2,5-dioctyloxy-p-phenylene vinylene)]/poly(ethylene oxide) blend; a poly[2-methoxy-5-(2′-ethylhexyloxy)-1,4-phenylene vinylene] (MEH-PPV)/polystyrene blend, a polyaniline/polystyrene blend; a polyaniline/polycarbonate blend, a poly(ethylene terephthalate)/poly(ethylene terephthalate)-co-poly(ethylene isophthalate) blend, a polysulfone/polyurethane blend; a chitosan/polylactide blend, a polyglycolide/chitin blend, and a polylactide/poly(lactide-co-glycolide) blend.

Nonlimiting examples of such block copolymers systems include polylactide-b-poly(ethylene oxide) block copolymers; poly(lactide-co-glycolide)-b-poly(ethylene oxide) block copolymers; poly[(trimethylene carbonate)-b-(ε-caprolactone)] block copolymers; polystyrene-b-polydimethylsiloxane and polystyrene-b-polypropylene block copolymers; polystyrene-b-polybutadiene-b-polystyrene block copolymers and polystyrene-b-polyisoprene block copolymers.

Nonlimiting examples of blends of a polymer with an inorganic material from which fibers can be produced include: montmorillonite with polyamide 6, polyamide 6,6 and poly(vinyl alcohol), poly(methyl methacrylate), or polyurethane as the carrier material; a blend of a polymer carrier and noble metal nanoparticles, for instance poly(acrylonitrile)-co-poly(acrylic acid)/Pd; poly(ethylene oxide)/Au; polyvinylpyrrolidone/Ag; and poly(acrylonitrile)/Ag; a blend of a polymer carrier and magnetic nanoparticles, for instance poly(ethylene oxide) (or poly(vinyl alcohol))/Fe₃O₄, poly(ε-caprolactone)/FePt; polyurethane/MnZnNi; and poly(methyl methacrylate)/Co; a blend of a polymer and carbon nanotubes, for instance carbon nanotubes blended with poly(acrylonitrile), poly(ethylene oxide), poly(vinyl alcohol), polylactide, polycarbonate, polystyrene, polyurethane, or poly(methyl methacrylate); a blend of a polymer and a metal oxide or metal sulfide, for instance: polymer/TiO₂ wherein the polymer may for instance be with polyvinylpyrrolidone, poly(vinyl acetate), or poly(acrylonitrile); polymer/ZrO₂ wherein the polymer may for instance be polyvinylpyrrolidone, poly(vinyl acetate) and poly(vinyl alcohol); and blends of a polymer or polymers with: ZnO, CuO, NiO, CeO₂, Mn₃O₄, Mn₂O₃/Mn₃O₄, MoO₃, BaTiO₃, Y₂O₃, Gd₂O₃, Ta₂O₅, Co₃O₄, Ba_{0.6}Sr_{0.4}TiO₃, SiO₂, CdS, PbS, and Ag₂S.

The fibers of the inner and outer portions more typically comprise, or for instance consist of, a polymer which is the same or different and is independently selected from the following: poly(lactide), poly(glycolide), poly(lactide-co-glycolide) (PLGA) and polycaprolactone (PCL). Poly(lactide-co-glycolide) (PLGA) is particularly preferred because, by adjusting the ratio of lactide to glycolide, the rate of degradation of the copolymer may be tuned, to adjust the rate at which cells are released from the scaffold in vivo, into the tissue in which the scaffold is located.

A thermoresponsive polymer may advantageously be used to keep a scaffold of the invention in place once it has been administered into target tissue in a patient, as part of a hybrid composition of the invention, e.g. by injection or implantation. This is because a thermoresponsive polymer may change shape in vivo, after administration into the target tissue, in a way that causes the device to be retained in that tissue. Possibilities include the use of a thermoresponsive polymer which contracts in vivo to cause a change in the shape of the scaffold that causes the scaffold to anchor itself within the target tissue. Alternatively, a thermoresponsive polymer which expands in vivo can be used to expand the scaffold in situ in order to lodge itself within the target tissue and resist ejection. Thermoresponsive polymers may be introduced into electrospun polymer fibers by cospinning with a main polymer. Thus, a thermoresponsive polymer may be present in the fibers in addition to any of the polymers listed above, for instance in addition to PLGA.

Thus, the inner portion, the outer portion, or both of the inner and outer portions, of the scaffold of the invention, may comprise a thermoresponsive polymer. The thermoresponsive polymer may be present in fibers in each portion instead of, or in addition to any of the polymers listed above.

Polymers that show thermoresponsivity in water (in an aqueous environment in vivo) and which may therefore be employed include poly(N-isopropylacrylamide), poly[2-(dimethylamino)ethyl methacrylate] (pDMAEMA), hydroxypropylcellulose, poly(vinylcaprolactame) and polyvinyl methyl ether. Poly(N-acryloylglycinamide), ureido-functionalized polymers, copolymers from N-vinylimidazole and 1-vinyl-2-(hydroxylmethyl)imidazole, or copolymers from acrylamide and acrylonitrile may alternatively be employed as a thermoresponsive polymer.

For instance, when the inner portion comprises fibers, the fibers of the inner portion may comprise a thermoresponsive polymer (either instead of, or in addition to any of the polymers listed above). Alternatively, when the inner portion is a solid polymer core, the polymer in that core may comprise a thermoresponsive polymer (either instead of, or in addition to any of the polymers listed above).

Similarly, the fibers of the outer portion may comprise a thermoresponsive polymer (either instead of, or in addition to any of the polymers listed above).

In one embodiment, the scaffold of the invention is cylinder-shaped, wherein the scaffold comprises: (i) said inner portion, wherein the inner portion runs the length of the scaffold and comprises aligned polymer fibers twisted around one another; and (ii) said outer portion, wherein the outer portion is disposed around the inner portion and comprises a porous, nonwoven network of polymer fibers; wherein: the scaffold has a length of from 4 mm to 8 mm and a diameter of from 200 μm to 500 μm; and the polymer fibers in the inner and outer portions are fibers of poly(lactide-co-glycolide) (PLGA), poly(lactide), poly(glycolide) or polycaprolactone (PCL) having a mean diameter of from about 2 μm to about 5 μm. The polymer fibers in the inner and outer portions of this embodiment may for instance be fibers of poly(lactide-co-glycolide) (PLGA) having a mean diameter of from about 2 μm to about 5 μm. The polymer fibers in the inner and outer portions may for example be electrospun fibers of poly(lactide-co-glycolide) (PLGA) having a mean diameter of from about 2 μm to about 5 μm.

The molar ratio of lactide to glycolide in the PLGA of this and other embodiments of the scaffold (or scaffold precursor) described herein may for example be about 85:15 or lower. A greater amount of glycolide in the copolymer generally corresponds to a higher PLGA degradation rate in water, with the exception that there is an anomalously high peak in the degradation rate of PLGA at a molar ratio of 50:50 lactide:glycolide. The molar ratio of lactide to glycolide in the PLGA may therefore be about 50:50, or lower than 50:50, e.g. from about 50:50 to about 1:99. The molar ratio of lactide to glycolide in the PLGA may for example be about 50:50, or from about 75:25 to about 1:99. It may for instance be about 50:50, or from about 85:15 to about 1:99. This provides a relatively high degradation rate. In another embodiment, molar ratio of lactide to glycolide in the PLGA may for example be from about 75:25 to about 85:15.

The invention also provides a scaffold precursor.

The scaffold precursor may be used as a scaffold in its own right, i.e. it may be the scaffold component in a hybrid composition of the invention, i.e. together with cells or biomolecules in accordance with the invention. Thus, in one embodiment, the hybrid composition of the invention may be as defined anywhere herein except that the scaffold component in the hybrid composition is a scaffold precursor of the invention as defined herein.

Usually, however, the scaffold precursor of the invention is used in the process of the invention for producing a scaffold of the invention, hence the term scaffold precursor. In particular, the scaffold precursor of the invention is typically used in the process of the invention for producing an elongate scaffold of the invention which comprises an inner portion comprising a plurality of polymer fibers twisted around one another and an outer portion comprising a porous, nonwoven network of polymer fibers.

The scaffold precursor of the invention is an elongate strip which comprises a plurality of layers of polymer fibers, the plurality of layers comprising: a first region, comprising at least one first layer of nonwoven polymer fibers; and a second region, disposed on the first region, the second region comprising at least one layer comprising aligned polymer fibers which are orientated along the length of the strip. The aligned polymer fibers are typically orientated substantially parallel to the length of the strip, for instance parallel to the length of the strip.

The scaffold precursor of the invention may optionally further comprise a third region, which is disposed on the second region, which third region comprises at least one further layer of nonwoven polymer fibers. In other words, the second region which includes the at least one layer comprising aligned polymer fibers may be sandwiched between two regions (the first and third regions) each comprising at least one layer of nonwoven polymer fibers.

In a preferred embodiment, the plurality of layers comprises the first region, the second region and the third region. Thus, for the avoidance of doubt the invention further comprises a scaffold precursor which is an elongate strip comprising a plurality of layers of polymer fibers, the plurality of layers comprising: a first region, comprising at least one first layer of nonwoven polymer fibers; a second region, disposed on the first region, the second region comprising at least one layer comprising aligned polymer fibers which are orientated along the length of the strip; and a third region which is disposed on the second region, which third region comprises at least one further layer of nonwoven polymer fibers. The aligned polymer fibers in the second region may be orientated parallel to the length of the strip.

Typically, the plurality of layers in the scaffold precursor of the invention has a thickness (depth) of from about 30 μm to about 1000 μm. The plurality of layers in the scaffold precursor may for instance have a thickness (depth) of from about 30 μm to about 800 μm, for instance from about 40 μm to about 600 μm, or from about 50 μm to about 400 μm. The plurality of layers in the scaffold precursor may for example have a thickness (depth) of from about 50 μm to about 200 μm, or for instance from about 80 μm to about 120 μm.

Often, the plurality of layers in the scaffold precursor has a width of from about 1 to about 15 times the thickness (depth) of the scaffold precursor, for instance from about 1 to about 10 times the thickness (depth). The plurality of layers may for instance have a width of from about 1 to about 7 times the thickness (depth) of the scaffold precursor, for instance from about 1 to 4 times the thickness (depth). The plurality of layers in the scaffold precursor may for instance have a width of from about 100 μm to about 2 mm, for instance a width of from about 400 μm to about 1.6 mm, or for example a width of from about 800 μm to about 1.2 mm.

In addition, the plurality of layers in the scaffold precursor may have a length of at least 30 times the thickness (depth) of the scaffold precursor. The scaffold precursor may for instance have a length of at least 50 times the thickness (depth) of the scaffold precursor. The length of the plurality of layers in the scaffold precursor may for instance be at least 100 times the thickness (depth) of the scaffold precursor, or for example at least 1000 times the thickness (depth) of the scaffold precursor.

The length of the plurality of layers in the scaffold precursor may for instance be at least 30 times the width of the scaffold precursor, for instance at least 50 times the width of the scaffold precursor, for instance at least 100 times the width of the scaffold precursor, for instance at least 1000 times the width of the scaffold precursor, or for example at least 4000 times the width of the scaffold precursor.

The length of the plurality of layers of the scaffold precursor may for instance be at least 1 mm, for instance at least 3 mm, but it is more typically at least 10 mm, for instance at least 30 mm, or for instance at least 50 mm. It may for instance be at least 100 mm, for example at least 200 mm. It may for instance be at least 100 mm, for instance be at least 200 mm, for instance be at least 1000 mm, for example at least 4000 mm. When the scaffold precursor is employed in the process of the invention for producing a scaffold, the length is typically longer at first (e.g. at least 10 mm and usually even longer) and is shortened during that process. Indeed, the length is typically shortened after the scaffold precursor has been twisted along its length in accordance with the process, at which point it may be cut into a desired length for the scaffold.

The fiber in each first layer of nonwoven polymer fibers in the scaffold precursor and, when present, in each further layer of nonwoven polymer fibers, may not have any particular orientation to speak of, i.e. the fiber may be randomly-oriented or at least approaching random orientation.

The porosity and pore size of each first layer of nonwoven polymer fibers in the scaffold precursor and, when present, each further layer of nonwoven polymer fibers, may be as defined above for the porous, nonwoven network of polymer fibers in the outer portion of the scaffold of the invention. Thus, each first layer of nonwoven polymer fibers in the scaffold precursor and, when present, each further layer of nonwoven polymer fibers, may have a porosity of equal to or greater than 50%. Further, each first layer of nonwoven polymer fibers in the scaffold precursor and, when present, each further layer of nonwoven polymer fibers, may have a mean pore size of from 10 μm to 20 μm.

The porosity and pore size of each first layer of nonwoven polymer fibers in the scaffold precursor and, when present, each further layer of nonwoven polymer fibers, are generally greater than those of the at least one layer comprising aligned polymer fibers orientated along the length of the strip. Thus, in the scaffold precursor of the invention, each first layer of nonwoven polymer fibers and, when present, each further layer of nonwoven polymer fibers, typically has a mean pore size which is greater than the mean pore size of the at least one layer comprising aligned polymer fibers. Similarly, each first layer of nonwoven polymer fibers and, when present, each further layer of nonwoven polymer fibers, usually has a porosity which is greater than the porosity of the at least one layer comprising aligned polymer fibers.

The first region may comprise more than one layer of nonwoven polymer fibers, i.e. there may be more than one “first layer” of nonwoven polymer fibers. For instance, the first region may comprise two or more “first layers” of nonwoven polymer fibers with one or more different properties. The one or more different properties of the two or more “first layers” of nonwoven polymer fibers may for example include one or more of the following: different fiber diameters, different polymers in the fibers, different porosities, different mean pore sizes, and different degrees of fiber alignment. For instance, the first region may comprise a primary “first layer” of nonwoven polymer fibers and a secondary “first layer” of nonwoven polymer fibers, wherein the secondary “first layer” is disposed between the primary “first layer” and the second region of the scaffold precursor. The secondary “first layer” may have a property (for instance a mean porosity, a mean pore size or a degree of fiber alignment) which is intermediate between the same property in the primary “first layer” and the at least one layer in the second region which comprises aligned polymer fibers. One example is that the secondary “first layer” may have a degree of fiber alignment which is less than that of the at least one layer in the second region which comprises aligned polymer fibers, but greater than that of the primary “first layer” in the first region. In this way, a smoother transition between the properties of the layers in the first region and the second region may be provided.

Similarly, the third region, when present, may comprise more than one layer of nonwoven polymer fibers, i.e. there may be more than one “further layer” of nonwoven polymer fibers. For instance, the third region may comprise two or more “further layers” of nonwoven polymer fibers with one or more different properties. The one or more different properties of the two or more “further layers” of nonwoven polymer fibers may for example include one or more of the following: different fiber diameters, different polymers in the fibers, different porosities, different mean pore sizes, and different degrees of fiber alignment. For instance, the third region may comprise a primary “further layer” of nonwoven polymer fibers and a secondary “further layer” of nonwoven polymer fibers, wherein the secondary “further layer” is disposed between the primary “further layer” and the second region of the scaffold precursor. The secondary “further layer” may have a property (for instance a mean porosity, a mean pore size or a degree of fiber alignment) which is intermediate between the same property in the primary “further layer” and the at least one layer in the second region which comprises aligned polymer fibers. One example is that the secondary “further layer” may have a degree of fiber alignment which is less than that of the at least one layer in the second region which comprises aligned polymer fibers, but greater than that of the primary “further layer” in the third region. In this way, a smoother transition between the properties of the layers in the second region and the third region may be provided.

The polymer fibers in the first, second and optional third regions may independently comprise electrospun, melt-spun, dry-spun, wet-spun, or extruded fibers. Usually, however, the polymer fibers in the first, second and optional third regions are all electrospun fibers.

The polymer fibers in the first, second and optional third regions may comprise a polymer as defined above for the scaffold of the invention. The polymer fibers of the second region may comprise the same polymer as, or a different polymer from, the polymer fibers employed in the first region and, where present, the optional third region. Typically, however, the same polymer is employed for the fibers of the first, second and optional third regions.

Thus, the polymer fibers in each of the first, second and optional third regions of the scaffold precursor of the invention may comprise a polymer which is the same as, or different from, the polymer(s) in the other region(s), and is independently selected from the polymers listed hereinbefore in respect of the polymers for the fibers of the scaffold of the invention.

Typically, the polymer fibers in the first, second and optional third regions comprise a bioabsorbable polymer. For instance, the polymer fibers in the first, second and optional third regions may comprise, or for instance consist of, poly(lactide), poly(glycolide), poly(lactide-co-glycolide) (PLGA), or polycaprolactone (PCL).

In the scaffold precursor of the invention, the mean diameter of the polymer fibers is usually from about 500 nm to about 10 μm. Typically, the mean diameter of the polymer fibers is from about 1 μm to about 5 μm, for instance from about 2 μm to about 5 μm. The mean diameter of the polymer fibers may for example from about 2 μm to about 4 μm, for example about 3 μm. The relative standard deviation from the mean is typically less than or equal to 25%.

The polymer fibers of the second region may have the same mean diameter as, or a different mean diameter from, the polymer fibers employed in the first region and, where present, the optional third region. Typically, however, the same mean diameter is employed for the fibers of the first, second and optional third regions. Thus, the mean diameter of the polymer fibers in the first, second and optional third regions is usually from 500 nm to 10 μm, and may be as further defined above, for instance from 1 μm to 5 μm, for instance from about 2 μm to about 4 μm, or for example about 3 μm. The relative standard deviation from the mean is typically less than or equal to 25%.

In one preferred embodiment, therefore, the invention provides a scaffold precursor in which the plurality of layers comprises: a first layer comprising nonwoven polymer fibers; a second layer, disposed on the first layer, the second layer comprising aligned polymer fibers orientated along the length of the strip; and optionally, a third layer, disposed on the second layer, the third layer comprising nonwoven polymer fibers; wherein the plurality of layers has a thickness (depth) of from about 30 μm to about 1000 μm, a width of from about 60 μm to about 2 mm, and a length of at least 1 mm (although the length is typically greater than this, as further defined hereinbefore for the scaffold precursor of the invention); and the polymer fibers in the first, second and third layers are fibers of poly(lactide-co-glycolide) (PLGA), poly(lactide), poly(glycolide) or polycaprolactone (PCL) having a mean diameter of from 500 nm to 10 μm. The mean diameter of the polymer fibers in the first, second and third layers is more typically however from 1 μm to 5 μm. The relative standard deviation from the mean is usually less than or equal to 25%.

Often, the plurality of layers comprises: a first layer comprising nonwoven polymer fibers; a second layer, disposed on the first layer, the second layer comprising aligned polymer fibers orientated along the length of the strip; and a third layer, disposed on the second layer, the third layer comprising nonwoven polymer fibers; wherein the plurality of layers has a thickness (depth) of from about 50 μm to about 200 μm, a width of from about 100 μm to about 2 mm, and a length of at least 10 mm; and the polymer fibers in the first, second and third layers are electrospun fibers of poly(lactide-co-glycolide) (PLGA) having a mean diameter of from about 2 μm to about 5 μm. The relative standard deviation from the mean may be less than or equal to 25%.

The invention further provides a process for producing a scaffold, which process comprises twisting a scaffold precursor of the invention along its length. The scaffold precursor may be anywhere as defined herein. Thus, it is generally an elongate strip comprising a plurality of layers of polymer fibers, the plurality of layers comprising: a first region, comprising at least one first layer of nonwoven polymer fibers; a second region, disposed on the first region, the second region comprising at least one layer comprising aligned polymer fibers which are orientated along the length of the strip; and, typically, a third region which is disposed on the second region, which third region comprises at least one further layer of nonwoven polymer fibers.

The scaffold precursor which is employed in the process of the invention may be as further defined anywhere herein. Typically, for instance, the plurality of layers of the scaffold precursor has a thickness (depth) of from about 30 μm to about 1000 μm and a width of from about 60 μm to about 2 mm, and the twisting is performed until the scaffold has a diameter of from about 200 μm to about 500 μm, optionally from about 300 μm to about 400 μm. The plurality of layers of the scaffold precursor may for instance have a thickness (depth) of from about 50 μm to about 200 μm and a width of from about 100 μm to about 2 mm, and the twisting is performed until the scaffold has a diameter of from about 200 μm to about 500 μm, optionally from about 300 μm to about 400 μm. Alternatively, the plurality of layers of the scaffold precursor may have a thickness (depth) of from about 80 μm to about 120 μm and a width of from about 800 μm to about 1.2 mm, and the twisting is performed until the scaffold has a diameter of from about 200 μm to about 500 μm, optionally from about 300 μm to about 400 μm. The length of the scaffold precursor employed in the process of the invention is typically at least 10 mm, for instance at least 30 mm, or for instance at least 50 mm. It may for instance be at least 100 mm, for example at least 200 mm.

Often, the process does not merely comprise twisting the scaffold precursor of the invention along its length. Rather, it often comprises: (i) treating the scaffold precursor with a solvent, and (ii) twisting the treated scaffold precursor along its length. It may further comprise: (iii) annealing the scaffold.

The solvent employed in step (i) typically comprises water. It often further comprises an alcohol. Thus, the solvent is often a mixture of water and the alcohol. The alcohol may be ethanol. The solvent may for instance be an aqueous solution of ethanol at a concentration of 30% (w/w).

The twisting may be performed until the scaffold has a particular diameter. Thus, the twisting is often performed until the scaffold has a diameter of from about 100 μm to about 1000 μm, for instance from about 150 μm to about 750 μm, or for example from about 200 μm to about 600 μm. The twisting may for instance be performed until the scaffold has a diameter of from about 200 μm to about 500 μm, or for example from about 300 μm to about 400 μm.

Typically, the twisting is performed until the scaffold has an average number of twists per mm of length of the scaffold of from 0.1 to 4. Often, the twisting is performed until the scaffold has an average number of twists per mm of length of the scaffold of from 0.3 to 3, for instance from 0.5 to 2.0, for example about 1. The twisting may for instance be performed until the scaffold has an average number of twists per mm of length of the scaffold of 1.0.

The process typically further comprises cutting the twisted scaffold precursor to a particular length. The particular length is typically at least about 2 times the diameter of the twisted scaffold precursor. More typically, the particular length is at least about 5 times the diameter of the scaffold, or for instance at least about 10 times the diameter of the scaffold. It may for example be at least about 50 times the diameter of the scaffold, for example at least about 100 times the diameter of the scaffold, for instance at least about 500 times the diameter of the scaffold, for instance at least 1000 times the width of the scaffold precursor, or for example at least 4000 times the width of the scaffold precursor.

Often, the process further comprises cutting the twisted scaffold precursor to a particular length which is from about 2 to about 1000 times the diameter of the scaffold, and is more typically from about 5 to about 1000 times the diameter of the scaffold, for instance from about 8 to about 1000 times the diameter of the scaffold. The particular length may for example be from about 8 to about 800 times the diameter of the scaffold, e.g. from about 10 to about 500 times the diameter of the scaffold, or from about 10 to about 300 times the diameter of the scaffold. The particular length to which the scaffold is cut may for instance be from about 10 to about 50 times the diameter of the scaffold, or from about 10 to about 30 times the diameter of the scaffold.

Thus, the process may further comprise cutting the twisted scaffold precursor to a particular length which is from about 1 mm to about 200 mm, for instance from about 2 mm to about 100 mm. The particular length may for instance be from about 4 mm to about 80 mm, or for example from about 5 mm to about 60 mm. The particular length may for example be from about 1 mm to about 10 mm, for instance from about 2 mm to about 10 mm, or, for instance, from about 4 mm to about 8 mm, for example from about 4 mm to about 6 mm.

Where the process of the invention comprises an annealing step (iii) as defined above, the annealing may take place either before or after the step of cutting the twisted scaffold precursor to a particular length.

The process of the invention may further comprise producing the scaffold precursor by:

(i) producing at least one first layer of nonwoven polymer fibers disposed on a collection substrate by electrospinning a fiber precursor solution onto a collection substrate while rotating the collection substrate at a first speed, wherein the fiber precursor solution comprises a polymer dissolved in a solvent;

(ii) producing at least one layer which comprises aligned polymer fibers on the at least one first layer, by electrospinning a fiber precursor solution onto the at least one first layer disposed on the collection substrate while rotating the collection substrate at a second speed which is greater than the first speed, wherein the fiber precursor solution comprises a polymer dissolved in a solvent; and

(iii) optionally, producing at least one further layer of nonwoven polymer fibers on the at least one layer which comprises aligned polymer fibers, by electrospinning a fiber precursor solution onto the at least one strengthening layer while rotating the collection substrate at a third speed which is less than the second speed, wherein the nanofiber precursor solution comprises a polymer dissolved in a solvent;

thereby forming a plurality of layers of polymer fibers on the collection substrate.

The first and third speeds, which are the same or different, may for instance be from to 50 RPM to 1000 RPM, for instance from 70 RPM to 500 RPM, or for example from 80 RPM to 120 RPM. The second speed may be from to 1500 to 3500 RPM, for instance from 2000 to 3000 RPM, for example from 2400 to 2600 RPM.

Typically, producing the scaffold precursor further comprises: cutting the plurality of layers of polymer fibers into an elongate strip and thereby producing said scaffold precursor.

More typically, producing the scaffold precursor further comprises: drying the plurality of layers of polymer fibers to remove residual solvent; and cutting the plurality of layers of polymer fibers into an elongate strip and thereby producing said scaffold precursor.

Cutting the plurality of layers of polymer fibers into an elongate strip usually comprises cutting the plurality of layers of polymer fibers with a laser into an elongate strip, i.e. laser cutting the plurality of layers. The laser cutting may for instance be performed with a CO₂ laser cutting tool.

Typically, the cutting step comprises cutting the plurality of layers of polymer fibers into at least one elongate strip having a width as defined hereinbefore for the scaffold precursor of the invention, and thereby producing said scaffold precursor. The width may for instance be from about 100 μm to about 2 mm, for instance from about 400 μm to about 1.6 mm, or for example from about 800 μm to about 1.2 mm, for instance about 1 mm.

Typically, the electrospinning process further comprises: removing the scaffold precursor thus produced from the collection substrate. The collection substrate typically comprises a release paper sheet, aluminum foil, or a silicone-coated sheet.

In another preferred embodiment of the process of the invention, the plurality of layers in the scaffold precursor comprises:

a first layer comprising nonwoven polymer fibers;

a second layer, disposed on the first layer, the second layer comprising aligned polymer fibers orientated along the length of the strip; and

a third layer, disposed on the second layer, the third layer comprising nonwoven polymer fibers;

wherein the plurality of layers has a thickness (depth) of from about 50 μm to about 200 μm, a width of from about 100 μm to about 2 mm, and a length of at least 10 mm; and

the polymer fibers in the first, second and third layers are electrospun fibers of poly(lactide-co-glycolide) (PLGA) having a mean diameter of from about 2 μm to about 5 μm (typically wherein the relative standard deviation from the mean is less than or equal to 25%);

and the process further comprises producing said scaffold precursor by:

(i) producing the first layer comprising nonwoven polymer fibers by electrospinning a fiber precursor solution onto a collection substrate while rotating the collection substrate at a first speed, wherein the fiber precursor solution comprises said PLGA dissolved in a solvent;

(ii) producing the second layer which comprises aligned polymer fibers on the first layer, by electrospinning a fiber precursor solution onto the first layer disposed on the collection substrate while rotating the collection substrate at a second speed which is greater than the first speed, wherein the fiber precursor solution comprises said PLGA dissolved in a solvent;

(iii) producing the third layer comprising nonwoven polymer fibers on the second layer, by electrospinning a fiber precursor solution onto the second layer while rotating the collection substrate at a third speed which is less than the second speed, wherein the fiber precursor solution comprises said PLGA dissolved in a solvent;

thereby forming a plurality of layers of polymer fibers on the collection substrate, the plurality of layers having a thickness (depth) of from about 50 μm to about 200 μm;

(iv) optionally, drying the plurality of layers of polymer fibers to remove residual solvent; and

(v) cutting the plurality of layers of polymer fibers into an elongate strip having a width of from about 100 μm to about 2 mm and a length of at least 10 mm, and thereby producing said scaffold precursor.

The first and third speeds, which are the same or different, may for instance be from to 50 RPM to 1000 RPM, for instance from 70 RPM to 500 RPM, or for example from 80 RPM to 120 RPM. The second speed may be from to 1500 to 3500 RPM, for instance from 2000 to 3000 RPM, for example from 2400 to 2600 RPM.

The process of electrospinning per se is well known and is described for instance in the following review articles: Z.-M. Huang, et al., Composites Science and Technology 63 (2003) 2223-2253 and in Greiner and Wendorff, Angew. Chem. Int. Ed. 2007, 46, 5670-5703.

The electrospinning steps in the steps of the process of the invention which concern producing the scaffold precursor, i.e. the steps of (i) producing at least one first layer of nonwoven polymer fibers disposed on a collection substrate; (ii) producing at least one layer which comprises aligned polymer fibers on the at least one first layer; and (iii) optionally, producing at least one further layer of nonwoven polymer fibers on the at least one layer which comprises aligned polymer fibers, each comprise electrospinning a fiber precursor solution onto a collection substrate, or onto a preceding layer on the collection substrate, while rotating the collection substrate at a particular speed, wherein the fiber precursor solution comprises a particular desired polymer dissolved in a solvent.

Typically, in carrying out these steps, a polymer or polymer blend from which the fibrous network is to be produced is dissolved in an appropriate solvent until a homogeneous solution of the required concentration is obtained. The concentration of the polymer solution must generally be high enough to achieve adequate chain entanglements in order for a continuous fiber to be formed. The polymer solution is typically then loaded into a vessel, usually a syringe, connected to a conductive (typically metal) capillary. The capillary is connected to a high voltage (usually to the positive terminal of a high voltage DC power supply), at a fixed distance from an earthed collection device. The collection device may be metallic and is typically covered in a collection substrate onto which the fibers are deposited. The collection device is preferably rotatable, to ensure uniform deposition of the material. Fibers are typically produced by passing the polymer solution at a fixed flow rate through the metal capillary whilst applying a high voltage to the capillary in order to establish an electric field between the capillary and the collection device. The applied voltage should be high enough to overcome the surface tension of the polymer droplet at the tip of the capillary. As the charge builds at the surface of the droplet, the surface area has to increase to accommodate the additional charge, this occurs through the formation of a Taylor Cone from the droplet, from which a continuous fiber is extracted. As the fiber travels toward the grounded collector, the solvent rapidly evaporates, and the fiber is further elongated due to instabilities arising from the columbic repulsions of the surface charges on the jet. The instabilities in the jet resulting from the high charge density cause the jet to whip about rapidly resulting in a nano/micro diameter solid (dry) filament. The collector is rotated slowly (e.g. at a rate of around 100 RPM) if a random, nonwoven fibrous layer on the substrate is desired. Alternatively, the collector may be rotated at a higher speed (e.g. at a rate of around 2500 RPM) if a layer of aligned fibers is desired. A plurality of layers of different fiber alignments, ranging from randomly orientated nonwoven layers to layers of highly aligned fibers, may be deposited by changing the rotation speed during the deposition. After a fixed amount of material has been deposited to generate a layer or plurality of layers of a particular desired thickness, the layer or plurality of layers is dried in order to remove any residual solvent/moisture from the fibers. Typically, it is dried under vacuum, for instance for 24-48 hours at room temperature (approx. 25° C.).

Any suitable polymer may be employed in the fiber precursor solution or solutions used in the electrospinning process. The polymer employed may be any of the polymers listed above in relation to the scaffold and scaffold precursor of the invention, or any of the copolymers, blends of two or more polymers, and blends of a polymer with an inorganic material listed hereinbefore. All of those polymers, copolymers and blends can be used in an electrospinning process to produce a porous three-dimensional network of nanofibers, as detailed in Greiner and Wendorff, Angew. Chem. Int. Ed. 2007, 46, 5670-5703. The polymer used is generally of course a biocompatible polymer, and preferably a bioabsorbable polymer, and may for instance be a natural polymer or a synthetic polymer. In some embodiments, the polymer is PLGA, as discussed hereinbefore in relation to the scaffold and scaffold precursor of the invention.

Any suitable solvent may be employed in the nanofiber precursor solution. A wide range of solvents can be used in electrospinning, including for instance water and polar, nonpolar, protic, and aprotic organic solvents. The solvent is chosen to suit the polymer or blend employed, particularly so that a homogeneous solution of the required concentration of the polymer can be obtained. A mixture of chloroform and methanol (96:4) is especially suitable when the polymer is Poly(L-lactide-co-glycolide) (PLGA).

Typically, therefore, the solvent comprises chloroform and methanol.

The concentration of the polymer in the solution should be high enough to achieve adequate chain entanglements in order for a continuous fiber to be formed. Typically, the concentration of the polymer in said solvent is from about 3 wt. % to about 20 wt. %. The concentration of the polymer in said solvent may for instance be from about 4 wt. % to about 10 wt. %. For instance, the concentration of the polymer in said solvent may be about 4 wt. %, to about 8 wt. %. For instance, the concentration of the polymer in said solvent may be about 6 wt. %.

The fiber precursor solution employed in the process of the invention may be a solution of from 3 wt. % to 20 wt. % PLGA in an organic solvent, such as for instance a mixture of chloroform and methanol. The nanofiber precursor solution may for instance be a solution of from 4 wt. % to 10 wt. % PLGA in an organic solvent, such as a mixture of chloroform and methanol. In one embodiment, the nanofiber precursor solution is a solution of about 6 wt. % PLGA in an organic solvent. The organic solvent is typically a mixture of chloroform and methanol.

Typically, the dispensing capillary of the fiber forming module has an inner diameter of from about 0.5 mm to about 1.0 mm.

In order to ensure uniform deposition on the collection substrate, the electrospinning typically further comprises moving at least a portion of the fiber collection device relative to the fiber forming module during said deposition. Thus, usually, the electrospinning further comprises moving at least a portion of the fiber collection device during said deposition.

Typically, the fiber collection device comprises a rotatable portion for rotating the collection substrate as described hereinbefore in the process of the invention. Thus, usually, the electrospinning further comprises rotating the rotatable portion during said deposition. The rotatable portion is typically a rotatable drum. The rotatable drum is typically rotated at the first and third speeds as defined above, for deposition of the nonwoven polymer fiber layers in the first and (optional) third regions of the scaffold precursor, for instance at a rate of from to 50 RPM to 1000 RPM, for instance from 70 RPM to 500 RPM, or for example from 80 RPM to 120 RPM. The rotatable portion is also typically rotated at the second speed, i.e. from to 1500 to 3500 RPM, for instance from 2000 to 3000 RPM, or for example from 2400 to 2600 RPM, for deposition of the aligned polymer fibers in the second region.

Deposition of the plurality of layers of the scaffold precursor on the collection substrate is continued until a plurality of layers of a particular desired thickness has been obtained. This thickness of the plurality of layers may be as further defined hereinbefore for the scaffold precursor of the invention, and may for instance be from about 30 μm to about 1000 μm, or for instance from about 50 μm to about 200 μm, for example from about 80 μm to about 120 μm.

Thus, typically the step of feeding said fiber precursor solution through the dispensing capillary whilst applying said voltage is performed until the thickness of the plurality of layers of the scaffold precursor has a thickness (depth) of from about 30 μm to about 1000 μm, for instance from about 50 μm to about 200 μm, or for example from about 80 μm to about 120 μm.

Typically, the flow rate at which the fiber precursor solution is fed through the dispensing capillary is from 100 μL/hr to 3000 μL/hr. More typically, it is from 400 μL/hr to 2500 μL/hr, for instance about 2000 μL/hr.

The distance between the dispensing capillary and the collection substrate is typically from 200 mm to 400 mm. More typically, it is from 200 mm to 300 mm, for instance about 250 mm.

The voltage applied across the dispensing capillary and the fiber collection device is typically from 20 kV to 30 kV. More typically, it is from 23 kV to 27 kV, for instance about 25 kV.

Usually, the electrospinning is performed at a temperature of from 22° C. to 28° C. More typically, the electrospinning is performed at a temperature of from 23° C. to 27° C., for instance about 25° C.

Typically, the electrospinning is performed in air having a relative humidity of from 20% to 45%. The electrospinning may for instance be performed in air having a relative humidity of 35% to 45%, for instance about 40%.

The electrospinning process, of producing the scaffold precursor, may further comprise: drying the plurality of layers of polymer fibers thus produced to remove residual solvent; and cutting the plurality of layers of polymer fibers into an elongate strip and thereby producing said scaffold precursor. Typically, the scaffold precursor is dried under vacuum. Typically, the drying is done at room temperature under vacuum.

Typically, the electrospinning process further comprises: removing the scaffold precursor thus produced from the collection substrate. The collection substrate typically comprises a release paper sheet, aluminum foil, or a silicone-coated sheet.

In the process of the invention for producing a scaffold, the scaffold may be as further defined anywhere herein. The scaffold produced by the process of the invention is generally one which comprises an inner portion which comprises a plurality of polymer fibers twisted around one another, and an outer portion comprising a porous, nonwoven network of polymer fibers, wherein the packing density of the inner portion is greater than the packing density of the outer portion. This scaffold produced by the process of the invention may be as further defined anywhere herein.

Scaffolds of the invention where the inner portion is a solid polymer core may be produced by a process which is similar to that of the process of the invention except that the elongate scaffold precursor that is twisted along its length will have, instead of at least one layer of aligned polymer fibers in the second region, at least one solid polymer layer in the second region. The at least one solid polymer layer in the second region may have relatively little or no porosity, i.e. it may be a compact layer consisting essentially of, or consisting of, a polymer. The at least one solid polymer layer in the second region may for instance be nonporous or have a lower porosity than the porous network of fibers. The scaffold precursor is then twisted along its length in order to form a scaffold of the invention in which the inner portion is a solid core comprising a polymer. Furthermore, scaffolds of the invention in which an inner portion comprises a porous, nonwoven network of polymer fibers is disposed within a protective outer portion which may comprise aligned polymer fibers, can be produced by a process in which outer layers of aligned polymer fibers are electrospun around an inner porous, nonwoven network of polymer fibers.

The invention also provides a scaffold which is obtainable by the process of the invention for producing a scaffold.

The scaffold which is obtainable by the process of the invention may be as further defined anywhere herein for the scaffold of the invention. The process of the invention by which that scaffold is obtainable may also be as further defined herein for the process of the invention.

For instance, in one preferred embodiment, the invention provides a cylinder-shaped scaffold which is obtainable by:

(i) treating a scaffold precursor with a solvent, wherein the scaffold precursor is an elongate strip comprising a plurality of layers of polymer fibers, the plurality of layers comprising:

a first layer comprising nonwoven polymer fibers;

a second layer, disposed on the first layer, the second layer comprising aligned polymer fibers orientated along the length of the strip; and

a third layer, disposed on the second layer, the third layer comprising nonwoven polymer fibers;

wherein the plurality of layers has a thickness (depth) of from about 50 μm to about 200 μm, a width of from about 100 μm to about 2 mm, and a length of at least 10 mm; and

the polymer fibers in the first, second and third layers are electrospun fibers of poly(lactide-co-glycolide) (PLGA) having a mean diameter of from about 2 μm to about 5 μm;

(ii) twisting the treated scaffold precursor along its length until the scaffold precursor has a diameter of from about 200 μm to about 500 μm; and

(iii) cutting the twisted scaffold precursor to a length of from about 4 mm to about 8 mm.

The invention also provides a scaffold which is obtained by the process of the invention for producing a scaffold. The scaffold which is obtained by the process of the invention may be as further defined anywhere herein for the scaffold of the invention. The process of the invention by which that scaffold is obtained may also be as further defined herein for the process of the invention.

The invention further provides a hybrid composition comprising: (i) cells, a biomolecule or other active agent; and (ii) a scaffold of the invention, a scaffold precursor of the invention, or a scaffold which is obtainable by or obtained by a process of the invention for producing a scaffold. The biomolecule or other active agent may be a drug, a nucleic acid, a nucleotide, a protein, a polypeptide, or an exosome. The nucleic acid may comprise DNA, RNA, RNAi, SaRNA or SiRNA.

Thus, in one embodiment, the invention further provides a hybrid composition comprising: (i) adherent therapeutic cells, a drug, a nucleic acid, a nucleotide, a protein, a polypeptide or an exosome, optionally wherein the nucleic acid comprises DNA, RNA, RNAi, SaRNA or SiRNA; and (ii) a scaffold of the invention, a scaffold precursor of the invention, or a scaffold which is obtainable by or obtained by a process of the invention for producing a scaffold.

Typically, the hybrid composition comprises: (i) cells, a biomolecule or other active agent; and (ii) a scaffold of the invention or a scaffold which is obtainable by or obtained by a process of the invention for producing a scaffold.

Often, the hybrid composition comprises: (i) cells; and (ii) a scaffold of the invention, a scaffold precursor of the invention, or a scaffold which is obtainable by or obtained by a process of the invention for producing a scaffold. Cells are not only capable of infiltrating into and growing within the porous network of fibers in the scaffold or scaffold precursor of the invention, in order to form a hybrid composition comprising the cells and the scaffold or scaffold precursor, but cells are also capable of adhering to and growing on the surface of the scaffold or scaffold precursor of the invention in order to form a hybrid composition. Accordingly, the cells may be disposed within the porous network of fibers in the scaffold or scaffold precursor. The cells may be disposed in pores of the scaffold or scaffold precursor. The cells may attach to the outer surface of the scaffold or scaffold precursor. The cells may be disposed on the outer surface of the scaffold or scaffold precursor. The cells may be disposed in pores of the scaffold or scaffold precursor and on the outer surface of the scaffold or scaffold precursor. The cells may be disposed in pores of the scaffold of the invention. The cells may attach to the outer surface of the scaffold of the invention. The cells may be disposed in pores of the scaffold of the invention and on the outer surface of the scaffold of the invention.

For instance, the hybrid composition may comprise: (i) cells, and (ii) a scaffold of the invention, or a scaffold which is obtainable by or obtained by a process of the invention for producing a scaffold. The cells may comprise adherent therapeutic cells, for instance stem cells. The adherent therapeutic cells may for example comprise progenitor cells of mesodermal lineage (PMLs), immunomodulatory progenitor (iMP) cells, immune-oncology mesodermal progenitor cells (ioMP) cells or a combination thereof.

The hybrid composition may for instance comprise (i) adherent therapeutic cells and (ii) a scaffold of the invention, or a scaffold which is obtainable by or obtained by a process of the invention for producing a scaffold; wherein the adherent therapeutic cells are disposed in pores of the scaffold, on the outer surface of the scaffold, or both. The adherent therapeutic cells may for instance comprise stem cells, for example progenitor cells of mesodermal lineage (PMLs), immunomodulatory progenitor (iMP) cells, immune-oncology mesodermal progenitor cells (ioMP) cells or a combination thereof.

The hybrid composition may for instance comprise (i) adherent therapeutic cells and (ii) a scaffold of the invention or a scaffold which is obtainable by or obtained by a process of the invention for producing a scaffold. The adherent therapeutic cells may be disposed in pores of the scaffold and/or may be disposed on the surface of (e.g. may adhere to the surface of) the scaffold. The adherent therapeutic cells may for instance comprise stem cells, for example progenitor cells of mesodermal lineage (PMLs), immunomodulatory progenitor (iMP) cells, immune-oncology mesodermal progenitor cells (ioMP) cells or a combination thereof.

In the hybrid composition of the invention, the scaffold of the invention or the scaffold which is obtainable by or obtained by a process of the invention for producing a scaffold, may be as further defined anywhere herein.

For instance, the scaffold of the invention may be cylinder-shaped, have a length of from about 4 mm to about 8 mm, and a diameter of from about 200 μm to about 500 μm. The polymer fibers in the first and second portions, or the inner and outer portions, of the scaffold of the invention, may for instance have a mean diameter of from about 2 μm to about 5 μm.

The cells employed in the hybrid composition of the invention may, be disposed in pores of the scaffold, on the outer surface of the scaffold, or both.

Typically, the cells may be genetically modified cells and/or stem cells.

Often, the cells in the hybrid composition of the invention comprise adherent therapeutic cells. Adherent cells are cells which are capable of adhering to culture vessels which have been specifically treated for the culture of adherent cells. The concept of adherent cells is well known to a person skilled in the art. The skilled person is capable of identifying whether or not cells are adherent. Therapeutic cells are cells which are capable of having a therapeutic effect. Therapeutic cells are typically living cells. Therapeutic cells are typically cells which are capable of repairing damaged or diseased tissue. The therapeutic cells are preferably autologous. In other words, the cells are preferably derived from the patient into which the cells will be administered to repair damaged or diseased tissue. Alternatively, the cells are preferably allogeneic. In other words, the cells are preferably derived from a patient that is immunologically compatible with the patient into which the cells will be administered to repair damaged or diseased tissue. The cells may be semiallogeneic. Semiallogeneic populations are typically produced from two or more patients that are immunologically compatible with the patient into which the cells will be administered. In other words, all of the cells are preferably genetically identical with the patient into which they will be administered or sufficiently genetically identical that the cells are immunologically compatible with the patient into which they will be administered.

The composition typically comprises more than one cell, such at least about 2, at least about 5, at least about 10, at least about 20, at least about 30, at least about 40, at least about 50, at least about 100, at least about 200, at least about 500, at least about 1000, at least about 2000, at least about 5000, at least about 10,000, at least about 50,000, at least about 100,000, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸ or at least about 2×10⁸ cells. In some instances, the composition may comprise at least 1.0×10⁷, at least 1.0×10⁸, at least 1.0×10⁹, at least 1.0×10¹⁰, at least 1.0×10¹¹, or at least 1.0×10¹² cells or even more cells.

The number of cells in the composition typically depends on the size of the shape. The number of cells is often from about 0.5×10⁵ to about 3×10⁵, such as from about 1×10⁵ to about 2×10⁵, particularly in a scaffold of the invention which is cylinder-shaped, and has a length of from about 4 mm to about 8 mm, and a diameter of from about 200 μm to about 500 μm.

The adherent therapeutic cells may comprise progenitor cells of mesodermal lineage (PMLs), immunomodulatory progenitor (iMP) cells, immune-oncology mesodermal progenitor cells (ioMP) cells or a combination thereof.

The PMLs may express detectable levels of CD62P (P-selectin) and/or CD62E (E-selectin).

The PMLs may for instance:
express detectable levels of CD50 (ICAM-3), CD62L (L-selectin), CD62P (P-selectin), CD102 (ICAM-2) and CD126 (IL-6R1); or
(a) express detectable levels of CD29, CD44, CD73, CD90, CD105, CD271, CD120a (tumor necrosis factor [TNF]-alpha Receptor 1), CD120b (TNF-alpha Receptor 2), CD50 (Intercellular Adhesion Molecule-3, ICAM-3), CD54 (ICAM-1), CD58 (Lymphocyte function-associated antigen-1, LFA-1), CD62E (E-selectin), CD62L (L-selectin), CD62P (P-selectin), CD106 (Vascular cell adhesion protein, VCAM-1), CD102 (ICAM-2), CD166 (Activated leukocyte cell adhesion molecule), CD104 (Beta 4 integrin), CD123 (Interleukin-3 Receptor), CD124 (Interleukin-4 Receptor), CD126 (Interleukin-6 Receptor), CD127 (Interleukin-7 Receptor) and fibroblast growth factor receptor (FGFR) and (b) do not express detectable levels of CD14, CD34 and CD45.

The iMP cells may express detectable levels of MIC A/B, CD304 (Neuropilin 1) and CD99. The iMP cells may for instance express detectable levels of MIC A/B, CD304 (Neuropilin 1), CD178 (FAS ligand), CD289 (Toll-like receptor 9), CD363 (Sphingosine-1-phosphate receptor 1), CD99, CD181 (C—X—C chemokine receptor type 1; CXCR1), epidermal growth factor receptor (EGF-R), CXCR2 and CD126. The iMP cells may further express detectable levels of one or more of CD3, CD3e, CD8, CD8b, CD4, CD5, CD6 and CD7.

The ioMP cells may express detectable levels of CD66e, CD121b, CD122, CD164, CD172a, CD203c, CD264, CD270, CD328, CD358, T cell receptor (TCR) gamma delta, FMC7 and ITGB7. ioMP cells do not express detectable levels of HLA-ABC, MIC A/B, Notch2, CD360, CLIP, and CD11b.

The adherent therapeutic cells may be autologous or allogeneic.

The cells in the hybrid composition of the invention may for instance be therapeutic cells which are: mesenchymal stem cells (MSCs). Suitable MSCs are known in the art. The therapeutic cells are preferably mesenchymal precursor cells (MPCs). The therapeutic cells are more preferably human mesenchymal precursor cells (MPCs).

The cells in the hybrid composition of the invention may alternatively be therapeutic cells which are: dendritic cells, platelets, fibroblasts or myofibroblasts.

The invention additionally provides a process for producing a hybrid composition of the invention as defined herein, comprising combining (i) a scaffold of the invention, a scaffold precursor of the invention, or a scaffold which is obtainable by or obtained by a process of the invention for producing a scaffold, and (ii) cells, a biomolecule or other active agent, in a culture vessel. Both (i) the scaffold, and (ii) the cells, biomolecule, or other active agent, may be as further defined anywhere herein.

In one embodiment, the process for producing a hybrid composition of the invention comprises combining (i) a scaffold of the invention, a scaffold precursor of the invention, or a scaffold which is obtainable by or obtained by a process of the invention for producing a scaffold, and (ii) adherent therapeutic cells, a drug, a nucleic acid, a nucleotide, a protein, a polypeptide or an exosome in a culture vessel.

The process for producing a hybrid composition of the invention may for instance comprise: (i) combining a scaffold of the invention as defined herein, a scaffold precursor of the invention, or a scaffold which is obtainable by or obtained by a process of the invention for producing a scaffold, and adherent therapeutic cells, in a culture vessel which is not modified to facilitate the attachment of cells; and (ii) allowing the adherent therapeutic cells to infiltrate and proliferate on the surface and within the outer portion of the scaffold and thereby producing said hybrid composition.

The number of the cells added to the vessel typically corresponds to the number of cells which should be present in the composition of the invention. The proportion of added cells which attach to the scaffold can be measured by removing the scaffold from the vessel and determining how many, if any, cells remain in the vessel. Techniques for culturing cells are well known to a person skilled in the art.

The scaffold and cells may be combined in any suitable culture vessel. The vessel may be a flask or a well of a flat plate, such as a standard 6-well, 24-well or 96-well plate. Such flasks and plates are commercially available from Corning, Fisher scientific, VWR suppliers, Nunc, Starstedt or Falcon.

The flask, vessel or well within which the scaffold and cells are combined is not modified to facilitate attachment or immobilization of the cells onto its surface. So-called “nontissue culture treated” flasks, vessels and wells are known in the art and commercially available from the sources above. The surfaces of such flasks, vessels and wells are not coated with molecules or otherwise chemically modified to bind adherent cells and immobilize or capture them on their surfaces. It is straightforward for the skilled person to determine whether or not a flask, vessel or well is modified to facilitate the attachment of cells by introducing adherent cells and seeing if they stick to the culture surface.

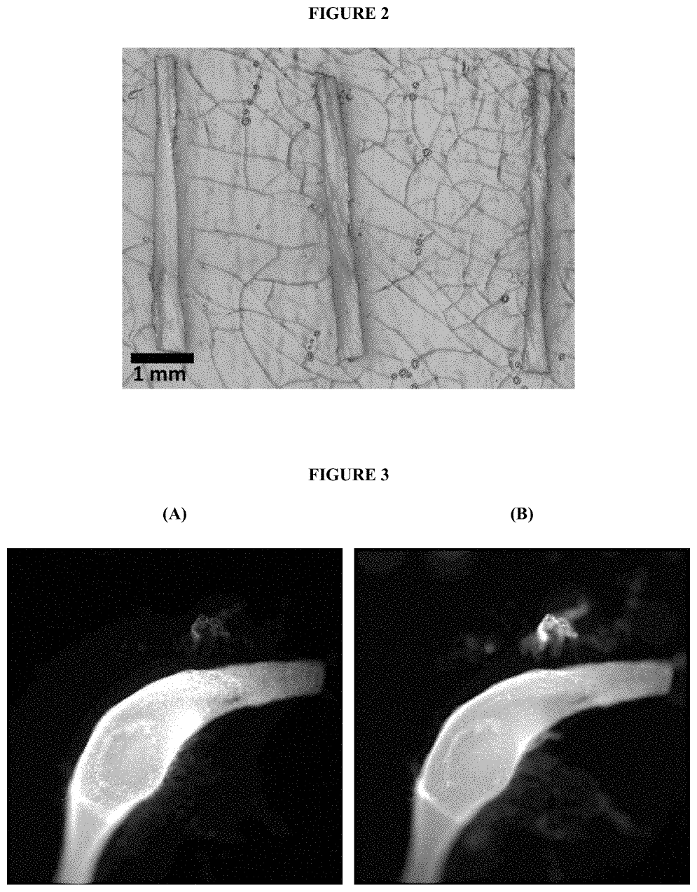

The hybrid composition of the invention is self-forming. Step (ii) of the method therefore comprises allowing the adherent therapeutic cells to attach to the scaffold. An example of this is shown in FIG. 3.

Step (ii) may take any amount of time. Step (ii) typically takes less than about 24 hours, such as less than about 18 hours, less than about 12 hours or less than about 8 hours. Step (ii) typically takes from about 1 hour to about 24 hours, such as from about 2 hours to about 18 hours, from about 3 hours to about 12 hours, from about 4 hours to about 10 hours or from about 5 hours to about 8 hours. Step (ii) typically takes about 6 hours. After the cells have attached to the scaffold, the composition may remain in culture, for instance up to about 48 hours, up to about 72 hours or up to about 96 hours after combination of the scaffold and cells.

The three-dimensional shape may alter its shape during step (ii). For instance, a straight scaffold may be curved during step (ii).

Steps (i) and (ii) typically involves culturing the cells in the presence of the scaffold. Techniques for culturing cells are well known to a person skilled in the art. The cells are may be cultured under standard conditions of 37° C. in medium without serum. The medium is preferably Minimum Essential Medium (MEM). MEM is commercially available from various sources including Sigma-Aldrich. The medium preferably further comprises heparin and/or L-glutamine. The L-glutamine may be replaced with GlutaMAX® (which is commercially available from Life Technologies).

The cells are preferably cultured with plasma lysate. Platelet lysate refers to the combination of natural growth factors contained in platelets that has been released through lysing those platelets. Lysis can be accomplished through chemical means (i.e. CaCl₂), osmotic means (use of distilled H₂O) or through freezing/thawing procedures. Platelet lysate can be derived from whole blood as described in U.S. Pat. No. 5,198,357. Platelet lysate is preferably prepared as described in International Patent Application No. PCT/GB2012/052911 (published as WO/2013/076507). For instance, it may be prepared by subjecting a population of platelets to at least one freeze-thaw cycle, wherein the freeze portion of each cycle is carried out at a temperature lower than or equal to −78° C. or −196° C. The platelet lysate is preferably prepared by subjecting a population of platelets to four freeze-thaw cycles, wherein the freeze portion of each cycle is carried out at a temperature lower than or equal to −78° C. or −196° C., for instance using liquid nitrogen.

The cells are preferably cultured under low oxygen conditions. The cells are preferably cultured at less than about 20% oxygen (02), such as less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2% or less than about 1% oxygen (02). The cells are preferably cultured at from about 0% to about 19% 02, such as from about 1% to about 15% 02, from about 2% to about 10% 02 or from about 5% to about 8% 02. The cells are most preferably cultured at about 0% 02. The figures for % oxygen (or % 02) quoted above relate to % by volume of oxygen in the gas supplied to the cells during culture, for instance by the cell incubator. It is possible that some oxygen may leak into the incubator or enter when the door is opened.

The cells are most preferably cultured in the presence of platelet lysate and under low oxygen conditions.

iMP cells expressing detectable levels of one or more of CD3, CD3e, CD8, CD8b, CD4, CD5, CD6, and CD7 may be cultured under any of the conditions disclosed in in International Patent Application No. PCT/GB2017/050917. ioMP cells may be cultured under any of the conditions described in International Patent Application No. PCT/GB2016/052447 (published as WO/2017/025729).

The invention further provides a hybrid composition of the invention for use in a method for treatment of the human or animal body by therapy.

The hybrid composition of the invention may be used in a method of therapy of the human or animal body. In particular, the invention concerns using the hybrid composition of the invention to repair a damaged or diseased tissue in a patient. The invention also concerns using a hybrid composition of the invention to treat a cardiac, bone, cartilage, tendon, ligament, liver, kidney joint, spleen, eye, spinal disc, connective tissue, or lung injury or disease in a patient or as a tissue filler for a reconstructive or cosmetic procedure. The invention also concerns using a hybrid composition of the invention to treat an infection or cancer in the patient.

The invention provides a method of repairing a damaged or diseased tissue in a patient, comprising contacting the damaged or diseased tissue with a hybrid composition of the invention, wherein the composition comprises a therapeutically effective number of cells, and thereby treating the damaged or diseased tissue in the patient. The invention also provides a hybrid composition of the invention for use in repairing a damaged or diseased tissue in the patient. The invention also provides use of a hybrid composition of the invention in the manufacture of a medicament for repairing a damaged or diseased tissue in a patient.

The damage to the tissue may be caused by injury or disease. The injury or disease is preferably a cardiac, bone, cartilage, tendon, ligament, liver, kidney joint, spleen, eye, spinal disc, connective tissue, or lung injury or disease in a patient. The invention also provides a hybrid composition of the invention for use as a tissue filler in reconstructive and cosmetic applications.

The invention therefore provides a method of treating a cardiac, bone, cartilage, tendon, ligament, liver, kidney joint, spleen, eye, spinal disc, connective tissue, or lung injury or disease in a patient, comprising administering to the patient a hybrid composition of the invention, wherein the composition comprises a therapeutically effective number of cells, and thereby treating the injury or disease in the patient. The invention also provides a reconstructive or cosmetic method comprising administering to the patient a hybrid composition of the invention as a tissue filler. The invention also provides a hybrid composition of the invention for use in treating a cardiac, bone, cartilage, tendon, ligament, liver, kidney joint, spleen, eye, spinal disc, connective tissue, or lung injury or disease in a patient, or for use as a tissue filler in a reconstructive or cosmetic application. The invention also provides use of a hybrid composition of the invention in the manufacture of a medicament for treating a cardiac, bone, cartilage, tendon, ligament, liver, kidney joint, spleen, eye, spinal disc, connective tissue, or lung injury or disease in a patient, or in the manufacture of a tissue filler for a reconstructive or cosmetic application.

The cardiac injury or disease is preferably selected from myocardial infarct (MI), left ventricular hypertrophy, right ventricular hypertrophy, emboli, heart failure, congenital heart deficit, heart valve disease, arrhythmia, and myocarditis.

MI increases the levels of VEGF and EPO released by the myocardium. Furthermore, MI is associated with an inflammatory reaction and infarcted tissue also releases macrophage migration inhibitory factor (MIF), interleukin (IL-6) and KC/Gro-alpha. CCL7 (previously known as MCP3), CXCL1, CXCL2 are significantly upregulated in the heart following myocardial infarct (MI) and might be implicated in regulating engraftment and homing of MSCs to infarcted myocardium.

In a myocardial infarct mice model, IL-8 was shown to highly up-regulate gene expression primarily in the first 2 days post-MI. Remarkably, the increased IL-8 expression was located predominantly in the infarcted area and the border zone, and only to a far lesser degree in the spared myocardium. By activating CXCR2, MIF displays chemokine-like functions and acts as a major regulator of inflammatory cell recruitment and atherogenesis.

The bone disease or injury is preferably selected from fracture, Salter-Harris fracture, greenstick fracture, bone spur, craniosynostosis, Coffin-Lowry syndrome, fibrodysplasia ossificans progressive, fibrous dysplasia, Fong Disease (or Nail-patella syndrome), hypophosphatasia, Klippel-Feil syndrome, Metabolic Bone Disease, Nail-patella syndrome, osteoarthritis, osteitis deformans (or Paget's disease of bone), osteitis fibrosa cystica (or Osteitis fibrosa or Von Recklinghausen's disease of bone), osteitis pubis, condensing osteitis (or osteitis condensans), osteitis condensans ilii, osteochondritis dissecans, osteogenesis imperfecta, osteomalacia, osteomyelitis, osteopenia, osteopetrosis, osteoporosis, osteonecrosis, porotic hyperostosis, primary hyperparathyroidism, renal osteodystrophy, bone cancer, a bone lesion associated with metastatic cancer, Gorham Stout disease, primary hyperparathyroidism, periodontal disease, and aseptic loosening of joint replacements. The bone cancer can be Ewing sarcoma, multiple myeloma, osteosarcoma (giant tumor of the bone), osteochondroma or osteoclastoma. The metastatic cancer that results in a bone lesion can be breast cancer, prostate cancer, kidney cancer, lung cancer and/or adult T-cell leukemia.

The damage or disease may be an infection or cancer. The invention therefore provides a method of treating an infection in a patient, comprising administering to the patient a hybrid composition of the invention, wherein the composition comprises a therapeutically effective number of cells, and thereby treating the infection in the patient. The invention also provides a hybrid composition of the invention for use in treating an infection in a patient. The invention also provides use of a hybrid composition of the invention in the manufacture of a medicament for treating an infection in a patient. The hybrid composition of the invention may be administered directly into the infected cells.

The infection may be caused by any pathogenic agent. The pathogenic agent may be a bacterium, an archaeon, a fungus, or a virus. The bacterium may be Gram negative or Gram positive. The Gram positive bacterium is preferably from the genus Bacillus, Clostridium, Enterococcus, Mycobacterium, Staphylococcus or Streptococcus. The Gram positive bacterium may be from the genus Pasteurella or Nocardia. The Gram negative bacterium is preferably from the genus Aggregatibacter, Bacteroides, Bartonella, Brucella, Campylobacter, Chylamidia, Enterbacter, Francisella, Haemophilus, Heliobacter, Klebsiella, Legionella, Moraxella, Neisseria, Porphyromonas, Pseudomonas, Salmonella, Serratia, Stenotrophomonas, Vibrio or Yersinia. The Gram negative bacterium may be from the genus Escherichia or Pseudomonas. The bacterium may be from the genus Borrelia, Chlamydophila, Listeria, Mycoplasma, Proteus, or Treponema. The bacterium is preferably Aggregatibacter actinomycetemcomitans, Bacillus anthracis, Bacillus licheniformis, Bacteroides fragilis, Bartonella henselae, Bordetella pertussis, Borrelia burgdorferi, Brucella abortus, Campylobacter jejuni, Chlamydia trachomatis, Chlamydophila pneumoniae, Clostridium difficile, Clostridium perfringens, Enterobacter aerogenes, Enterococcus faecalis, Enterococcus faecium, Francisella tularensis, Haemophilus influenzae, Helicobacter pylori, Klebsiella oxytoca, Legionella pneumophila, Listeria monocytogenes, Moraxella catarrhalis, Mycobacterium avium, Mycobacterium bovis, Mycoplasma genitalium, Mycoplasma pneumoniae, Neisseria gonorrhoeae, Neisseria meningitidis, Porphyromonas gingivalis, Proteus mirabilis, Pseudomonas aeruginosa, Salmonella enter ica, Serratia marcescens, Staphylococcus aureus, Staphylococcus epidermidis, Staphylococcus haemolyticus, Stenotrophomonas maltophilia, Streptococcus mutans, Streptococcus pyogenes, Streptococcus salivarius, Streptococcus sanguinis, Treponema pallidum, Vibrio cholera, Vibrio parahaemolyticus or Yersinia enterocolitica. Other specific examples of bacteria include, but are not limited, to Mycobacterium tuberculosis, Mycobacterium intracellilare, Mycobacterium kansaii, Mycobacterium gordonae, Streptococcus agalactiae, Streptococcus viridans group, Streptococcus faecalis, Streptococcus bovis, Streptococcus pneumoniae, Corynebacterium diptheriae, Erysipelothrix rhusiopathie, Clostridium tetani, Klebsiella pneumoniae, Pasteurella multocida, Fusobacterium nucleatum, Streptobacillus moniliformis, Treponema pertenue, and Actinomyces israelii.

The fungus is preferably from the genus Absidia, Acremonium, Aspergillus, Aureobasidium, Basidiobolus, Blastomyces, Blastoschizomyces, Candida, Cladosporium, Coccidioides, Cryptococcus, Cunninghamella, Curvularia, Debaryomyces, Exophiala, Exserohilum, Fonsecea, Fusarium, Geotrichum, Histoplasma, Issatchenkia, Kluyveromyces, Malezzesia, Mucor, Paracoccidioides, Paecilomyces, Penicillium, Pichia, Pneumocystis, Rhizomucor, Rhizopus, Rhodotorula, Saccharomyces, Scedosporium, Schizophyllum, Scopulariopsis, Sporothrix, Trichoderma, Trichophyton, or Trichosporon.

The fungus is preferably Aspergillus fumigatus, Aspergillus flavus, Aspergillus lentulus, Aspergillus terreus, Aspergillus nidulans, Aspergillus oryzae, Aspergillus niger, Candida albicans, Candida caribbica (Candida fermentati), Candida dubliniensis, Candida famata (Debaryomyces hansenii), Candida fukuyamaensis (Candida xestobii or Candida carpophila), Candida guilliermondii, Candida kefyr (Kluyveromyces marxianus), Candida krusei (Issatchenkia orientalis), Candida metapsilosis, Candida orthopsilosis, Candida parapsilosis, Candida parapsilosis, Candida pelliculosa, Candida psychrophila, Candida rugosa, Candida smithsonii, Candida tropicalis, Candida utilis, Coccidioides immitis, Cryptococcus bacillisporus, Cryptococcus gattii, Cryptococcus grubii, Cryptococcus neoformans, Debaryomyces coudertii, Debaryomyces maramus, Debaryomyces nepalensis, Debaryomyces prosopidis, Debaryomyces robertsiae, Debaryomyces udenii, Histoplasma capsulatum, Kluyveromyces lactis, Pichia cecembensis, Rhodotorula araucariae, Rhodotorula babjevae, Rhodotorula dairensis, Rhodotorula diobovatum, Rhodotorula glutinis, Rhodotorula kratochvilovae, Rhodotorula paludigenum, Rhodotorula sphaerocarpum, Rhodotorula toruloides, Rhodotorula mucliaginosa, Saccharomyces “sensu stricto”, Saccharomyces bayanus, Saccharomyces boulardii, Saccharomyces cariocanus, Saccharomyces kudiavzevii, Saccharomyces mikatae, Saccharomyces paradioxus, Saccharomyces pastorianus, Saccharomyces uvarum, Saccharomyces cerevisiae or Tsuchiyaea wingfieldii.

The virus may belong to the family Retroviridae, such as human deficiency viruses, such as HIV-n (also referred to as HTLV-III), HIV-II, LAC, IDLV-III/LAV, HIV-III or other isolates such as HIV-LP, the family Picornaviridae, such as poliovirus, hepatitis A, enteroviruses, human Coxsackie viruses, rhinoviruses, echoviruses, the family Calciviridae, such as viruses that cause gastroenteritis, the family Togaviridae, such as equine encephalitis viruses and rubella viruses, the family Flaviviridae, such as dengue viruses, encephalitis viruses and yellow fever viruses, the family Coronaviridae, such as coronaviruses, the family Rhabdoviridae, such as vesicular stomata viruses and rabies viruses, the family Filoviridae, such as Ebola viruses, the family Paramyxoviridae, such as parainfluenza viruses, mumps viruses, measles virus and respiratory syncytial virus, the family Orthomyxoviridae, such as influenza viruses, the family Bungaviridae, such as Hataan viruses, bunga viruses, phleoboviruses and Nairo viruses, the family Arena viridae, such as hemorrhagic fever viruses, the family Reoviridae, such as reoviruses, orbiviruses and rotaviruses, the family Bimaviridae, the family Hepadnaviridae, such as hepatitis B virus, the family Parvoviridae, such as parvoviruses, the Papovaviridae, such as papilloma viruses and polyoma viruses, the family Adenoviridae, such as adenoviruses, the family Herpesviridae, such as herpes simplex virus (HSV) I and II, varicella zoster virus and pox viruses, or the family Iridoviridae, such as African swine fever virus). The virus may be an unclassified virus, such as the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses. The virus is preferably the influenza virus.

The invention therefore also provides a method of treating cancer in a patient, comprising administering to the patient a hybrid composition of the invention, wherein the composition comprises a therapeutically effective number of cells, and thereby treating the cancer in the patient. The invention also provides a hybrid composition of the invention for use in treating cancer in a patient. The invention also provides use of a hybrid composition of the invention in the manufacture of a medicament for cancer a patient. The hybrid composition of the invention may be administered directly into the cancer cells.

Preferably, the cancer is anal cancer, bile duct cancer (cholangiocarcinoma), bladder cancer, blood cancer, bone cancer, bowel cancer, brain tumors, breast cancer, colorectal cancer, cervical cancer, endocrine tumors, eye cancer (such as ocular melanoma), fallopian tube cancer, gall bladder cancer, head and/or neck cancer, Kaposi's sarcoma, kidney cancer, larynx cancer, leukemia, liver cancer, lung cancer, lymph node cancer, lymphoma, melanoma, mesothelioma, myeloma, neuroendocrine tumors, ovarian cancer, esophageal cancer, pancreatic cancer, penis cancer, primary peritoneal cancer, prostate cancer, Pseudomyxoma peritonei, skin cancer, small bowel cancer, soft tissue sarcoma, spinal cord tumors, stomach cancer, testicular cancer, thymus cancer, thyroid cancer, trachea cancer, unknown primary cancer, vagina cancer, vulva cancer or endometrial cancer. The leukemia is preferably acute lymphoblastic leukemia, acute myeloid leukemia, chronic lymphocytic leukemia, or chronic myeloid leukemia. The lymphoma is preferably Hodgkin lymphoma or non-Hodgkin lymphoma. The cancer is preferably primary cancer or secondary cancer.

iMP cells, iMP cells expressing one or more of CD3, CD3e, CD8, CD8b, CD4, CD5, CD6 and CD7 and ioMP cells are particularly suited to treating infections and cancer. These cells may be used alone as part of a hybrid composition of the invention. These cells may regulate the activity of any immune cells, such as T cells, B cells, dendritic cells, neutrophils, basophils, mast cells, eosinophils, innate lymphoid cells (ILCs), natural killer (NK) cells, monocytes, macrophages, megakaryocytes, thymocytes or platelets, in the patient. Preferably, the cells are used to regulate the activity of T cells. More preferably, the iMP cells are used to regulate the activity of helper T (Th) cells, cytotoxic T cells, regulatory T cells (Treg), gamma delta T cells or natural killer T (NKT) cells. Gamma delta T cells are preferred. Alternatively, the hybrid composition of the invention may be administered to the patient in combination with any of the immune cells listed above, preferably T cells or NK cells. Any of the therapeutic methods disclosed in International Patent Application No. PCT/GB2012/051600 (published as WO/2013/005053), International Patent Application No. PCT/GB2015/051673 (WO/2015/189587), International Patent Application No. PCT/GB2017/050917 or International Patent Application No. PCT/GB2016/052447 (published as WO/2017/025729) may be used in the context of this invention.

In all instances, the therapeutic cells are preferably derived from the patient or an allogeneic donor. Deriving the cells from the patient should ensure that the cells are themselves not rejected by the patient's immune system. Any difference between the donor and recipient will ultimately cause clearance of the cells, but not before they have repaired at least a part of the damaged or diseased tissue.

The invention concerns administering to the patient a therapeutically effective number of therapeutic cells to the patient. A therapeutically effective number is a number which ameliorates one or more symptoms of the damage, disease, or injury. A therapeutically effective number is preferably a number which repairs the damaged or diseased tissue or treats the disease or injury. Suitable numbers are discussed in more detail below.

The hybrid composition of the invention may be administered to any suitable patient. The patient is generally a human patient. The patient may be an infant, a juvenile or an adult. The patient may be known to have a damaged or diseased tissue or is suspected of having a damaged or diseased tissue. The patient may be susceptible to, or at risk from, the relevant disease or injury. For instance, the patient may be genetically predisposed to heart failure.

The invention may be used in combination with other means of, and substances for, repairing damaged or diseased tissue or providing pain relief. In some cases, the hybrid composition of the invention may be administered simultaneously, sequentially, or separately with other substances which are intended for repairing the damaged or diseased tissue or for providing pain relief. The hybrid composition of the invention may be used in combination with existing treatments for damaged or diseased tissue and may, for example, be simply mixed with such treatments. Thus, the invention may be used to increase the efficacy of existing treatments of damaged or diseased tissue.

The invention preferably concerns the use of therapeutic cells loaded or transfected with a therapeutic and/or diagnostic agent. A therapeutic agent may help to repair the damaged or diseased tissue. A diagnostic agent, such as a fluorescent molecule, may help to identify the location of the composition in the patient. The cells may be loaded or transfected using any method known in the art. The loading of cells may be performed in vitro or ex vivo. In each case, the cells may simply be in contact with the agent in culture. Alternatively, the cells may be loaded with an agent using delivery vehicle, such as liposomes. Such vehicles are known in the art.

The transfection of cells is well known in the art. The cells are typically transfected with a nucleic acid encoding the agent. For instance, viral particles or other vectors encoding the agent may be employed.

The nucleic acid gives rise to expression of the agent in the cells. The nucleic acid molecule will preferably comprise a promoter which is operably linked to the sequences encoding the agent and which is active or which can be induced in the cells.

In a particularly preferred embodiment, the nucleic acid encoding the agent may be delivered via a viral particle. The viral particle may comprise a targeting molecule to ensure efficient transfection. The targeting molecule will typically be provided wholly or partly on the surface of the virus in order for the molecule to be able to target the virus to the cells.

The hybrid composition of the invention may also be employed in wound healing. The invention thus further provides a method of treating (e.g. healing) a wound in a patient, comprising administering to the patient a hybrid composition of the invention, wherein the composition comprises a therapeutically effective number of cells, and thereby treating the wound in the patient. The invention also provides a hybrid composition of the invention for use in treating (e.g. healing) a wound. The invention also provides use of a hybrid composition of the invention in the manufacture of a medicament for treating (e.g. healing) a wound in a patient. Typically, the scaffold in the hybrid composition of the invention in this embodiment comprises at least one nonwoven layer of polymer fibers and cells. The cells include, for example, autologous or allogeneic cells, stem cells, or macerated tissue perioperatively, to aid in healing of the wound.

The hybrid composition of the invention may also be employed in fat transplantation and tissue filling procedures. Adipose tissue cells, fibroblasts, dermal stem cells, adipose tissue-derived mesenchymal stem cells, embryonic stem cells, induced stem cells (iSC) and progenitors derived from such cells (induced somatic stem cells) are coadministered with the scaffold, ideally once allowed to adhere to a scaffold of the invention to form a hybrid composition, and the hybrid composition is then administered, for example by injection, into connective tissue employed in cosmetic and reconstructive surgeries. Pores may be designed in the scaffold that preferentially attract stem cells, and current fat preparation systems may be used to “load” scaffolds of the invention via a port. Examples include the LifeCell Revolve system, Cytori Stemsource, puregraft system, and VASER Lipo system. The cells and devices may then be administered to the connective tissue together; ideally but not necessarily after waiting for adhesion to the devices.

The hybrid composition of the invention may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA.

The composition may be administered by any route. Suitable routes include, but are not limited to, intravenous, intramuscular, intraperitoneal, or other appropriate administration routes. The composition is preferably administered directly to the damaged or diseased tissue. By injection or insertion via a catheter are particularly preferred when the elongate (e.g. cylinder-shaped) scaffold of the invention is employed in the hybrid composition of the invention.

The composition of scaffold and therapeutic agent (the hybrid composition of the invention) may be administered by any route, as discussed above, but is preferably administered directly to the damaged or diseased tissue via a syringe, needle, or catheter. In order to minimize damage to the target tissue during insertion it is beneficial to use the smallest possible insertion device size. As a person skilled in the art will appreciate, this places constraints on the size and shape of the composition. Usually the scaffold of the invention is elongate, and can for example be cylinder-shaped, as this would facilitate delivery to the target tissue by the above-mentioned methods. Examples of injectable, cylinder-shaped, cell or therapeutic agent carrying devices include Ozurdex and Iluvien and are further exemplified in US 2008-0147021 A1, US 2014-0105956 A1, WO/2016/191645 A1 and WO/2017/089797 A1. However, any shape may in principle be employed.

Compositions may be prepared together with a physiologically acceptable carrier or diluent. The composition may be mixed with an excipient which is pharmaceutically acceptable and compatible with the active ingredient. Suitable excipients are, for example, water, saline, dextrose, glycerol, of the like and combinations thereof.

In addition, if desired, the pharmaceutical compositions of the invention may contain minor amounts of auxiliary substances such as wetting, gelling or emulsifying agents, pH buffering agents, and/or adjuvants which enhance effectiveness. The composition preferably comprises human serum albumin.

One suitable carrier or diluents is Plasma-Lyte A®. This is a sterile, nonpyrogenic isotonic solution for intravenous administration. Each 100 mL contains 526 mg of Sodium Chloride, USP (NaCl); 502 mg of Sodium Gluconate (C₆H₁₁NaO₇); 368 mg of Sodium Acetate Trihydrate, USP (C₂H₃NaO₂.3H₂O); 37 mg of Potassium Chloride, USP (KCl); and 30 mg of Magnesium Chloride, USP (MgCl₂.6H₂O). It contains no antimicrobial agents. The pH is adjusted with sodium hydroxide. The pH is 7.4 (6.5 to 8.0).

The composition is administered in a manner compatible with the dosage formulation and in such an amount that will be therapeutically effective. The quantity of the therapeutic cells to be administered depends on the subject to be treated, capacity of the subject's immune system and the degree repair desired. Precise amounts of cells required to be administered may depend on the judgement of the practitioner and may be specific to each subject.

Any suitable number of cells may be administered to a subject. For example, at least, or about, 0.2×10⁶, 0.25×10⁶, 0.5×10⁶, 1.5×10⁶, 4.0×10⁶, or 5.0×10⁶ cells per kg of patient may administered. For example, at least, or about, 10⁵, 10⁶, 10⁷, 10⁸, 10⁹ cells may be administered. As a guide, the number of cells to be administered may be from 10⁵ to 10⁹, preferably from 10⁶ to 10⁸. Any of the specific numbers discussed above may be administered.

The present invention is further illustrated in the Examples which follow.

### EXAMPLES

### Example 1: Scaffold Production

Poly(L-lactide-co-glycolide) (PLGA) (Purasorb, PLG8531, Corbion Purac, Netherlands) with an inherent viscosity midpoint of 3.1 dL/g was used in the manufacture of the scaffold precursor by electrospinning. A solution containing 6.0 wt. % of PLG8531 in chloroform and methanol (96:4) (Sigma-Aldrich, UK) was prepared.

The scaffold precursor contained three distinct layers of electrospun fiber: nonwoven top and bottom layers and a central layer containing uniaxially aligned electrospun fibers. The electrospun scaffold precursors were prepared by delivering the polymer solution at a constant feed rate of 2000 μL/h per needle via a syringe pump and was electrospun horizontally with an accelerating voltage of +25 kV DC on the needles of internal diameter of 0.8 mm, with PTFE tubing (inner diameter 1.6 mm). Temperature and relative humidity were kept constant (respectively at 25° C. and 40% RH) in a climate controlled electrospinning machine (EC-CLI, IME Technologies, The Netherlands). Fibers were collected on release paper sheets wrapped around a rotating collector (90 mm diameter) positioned 25 cm from the tip of the needle. The collector was rotated at 100 RPM when preparing the top and bottom nonwoven layers, and at 2500 RPM when preparing the central aligned fiber layer. Longitudinal translation was also applied, using a programmable motorized stage with a translation speed of 65 mm/s. Electrospinning was performed for 45 minutes to fabricate the desired sheet thickness, i.e. the desired thickness for the scaffold precursor.

Fiber diameter and scaffold morphology characterization were performed by scanning electronic microscopy (SEM) (Phenom G2 Pro equipped with FiberMetric Software, Phenom World, the Netherlands), using automated image characterization of multiple images in order to determine the mean fiber diameter and the relative standard deviation. The FiberMetric software automatically identifies the location of the fibers within the captured SEM image and measures the diameter of each fiber 20 times at a specific location. Typically, around 100 of such measurements are performed per image. The diameter of the fibers can alternatively be obtained via manual measurements/analysis of multiple SEM images.

The target average fiber diameter of the fibers was 3 μm with a tolerance of ±20%. Thickness of the sheet is measured using a micrometer. The target average thickness of the material was 100 μm with a tolerance of ±20%.

The fibrous mat was dried in a vacuum oven at ˜10 mbar for over 24 hours at 25° C. to reduce the amount of residual solvent remaining from the fabrication process.

The sheet of material was then laser cut using a CO₂ laser cutting tool (Mini 24, CSI Epilog, USA) into 1 mm wide strips (with the width being taken as the dimension perpendicular to the direction of fiber alignment in the central layer) to produce elongate scaffold precursors.

The scaffold precursor strips were then removed from the release paper and briefly soaked in a 30% ethanol (aqueous) solution. The wet strips were then manually twisted into cylinder-shaped scaffolds, to achieve a “cylinder” diameter of between 300-4000 microns, with an average of 1 twist per mm (measured with the twisted structure under tension). The twisted strips were then cut into 5 mm lengths, to produce 5 mm long cylinder-shaped scaffolds.

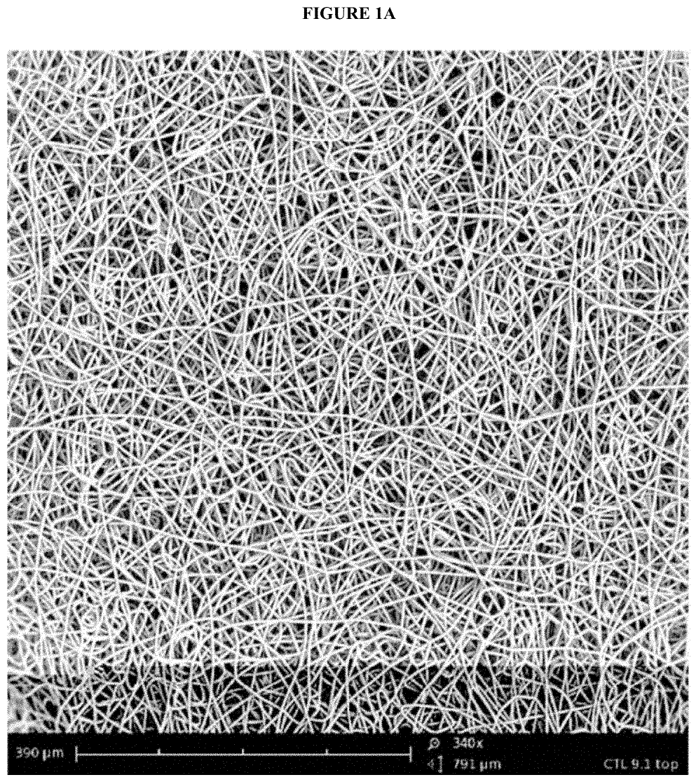

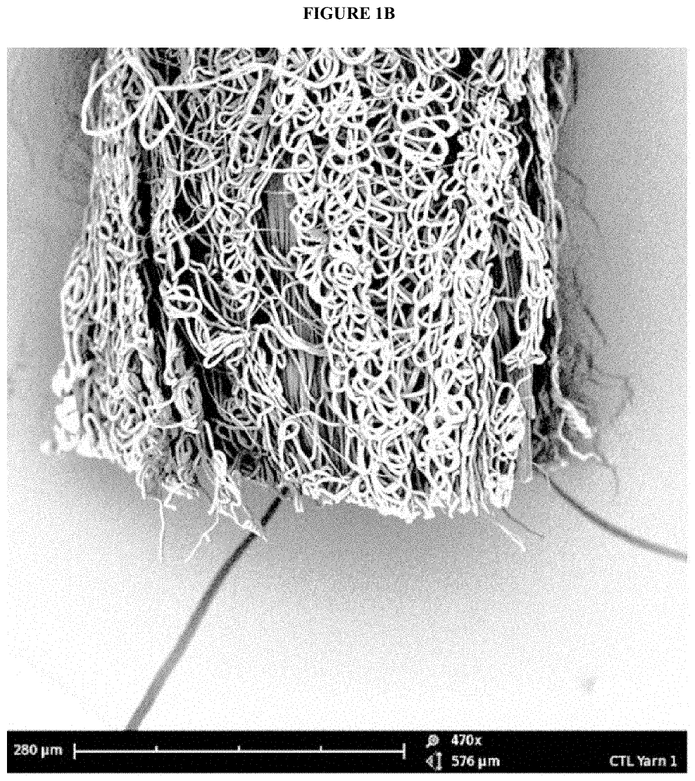

FIG. 1A is an SEM image of an outer, nonwoven layer of the trilayer sheet produced by electrospinning as described above, during the manufacture of the scaffold precursor. FIG. 1B shows one end of the PLGA cylinder-shaped scaffold that has been produced by twisting a trilayer scaffold precursor strip and then cutting as described above. FIG. 2 shows an optical microscope composite image of three PLGA cylinder-shaped scaffolds demonstrating different number of twists per unit length that can be achieved.

### Example 2: Production of a Hybrid Composition

Electrospun PLGA cylinder-shaped scaffolds were custom built in seven batches by The Electrospinning Company, Oxford UK, in accordance with the process described in Example 1. A scanning electron microscope image (SEM) of the end of a scaffold is shown in FIG. 1B and an electrospun sheet from which such a cylinder-shaped scaffold is formed is shown in FIG. 1A.

The specification of the cylinder-shaped scaffold was as follows.

Sheets were electrospun as described in Example 1 using PURASORB PDLG 8531, a GMP grade copolymer of DL-lactide and glycolide comprising an 85/15 molar ratio of lactide to glycolide and with an inherent viscosity midpoint of 3.1 dL/g. The sheets were formed into seven batches of scaffolds, as described in Example 1.

Scaffold length
4.96 mm±0.18 mm

Scaffold diameter
Batch I: 320 μm±60
Batch II: 400 μm±80
Batch III: 340 μm±70
Batch IV: 340 μm±60
Batch V: 310 μm±50
Batch VI: 380 μm±90
Batch VII: 300 μm±70

Fiber diameter
Batch I: 3.27 μm±0.87
Batch II: 3.17 μm±0.75
Batch III: 2.96 μm±0.90
Batch IV: 3.17 μm±0.76
Batch V: 2.90 μm±0.85
Batch VI: 3.06 μm±0.66
Batch VII: 3.05 μm±0.83

iMP cells were cultured by Cell Therapy Limited as described in International Patent Application No. PCT/GB2015/051673 (WO/2015/189587). The iMP cells were harvested and counted using a NucleoCounter 2×10⁵ iMP cells were placed in the well of a 96-well nontissue culture treated plate (Corning® catalog number 351172) with one of the scaffolds. The cells and scaffold were combined in culture medium as disclosed in International Patent Application No. PCT/GB2015/051673 (WO/2015/189587) and cultured under the conditions set out therein. In particular, the cells and scaffold were combined in 200 μL of cell culture media, αMEM, GlutaMAX, penicillin-streptomycin, platelet lysate, heparin and incubated at 37° C., 5% CO₂, 0% O₂.

The iMP cells began to attach to the scaffold within two hours. The scaffold was completely covered with cells within 6 to 8 hours. The experiment was repeated six times and the same results were achieved each time.

The number of cells on each scaffold was estimated as follows:
2×10⁵ cells were added to each well.
The hybrid composition was removed from the well.
The cells remaining in the well were counted using a NucleoCounter®.
The cell numbers present in the wells were below the detectable limit of the NucleoCounter® (below 1×10³ cells per well).
1×10³ cells are 1% of the total cells added to each well.
Approximately 99% of the cells attached to the scaffold.
Approximately 1.98×10⁵ cells per scaffold.

The hybrid composition of the invention remained in culture until 96 hours after combining the cells and scaffold.

The iMP cells were also imaged as follows:
1. After 96 hours, the scaffold was washed twice with 200 μL Hank's Buffered Salt Solution (HBSS).
2. The scaffold was fixed with 2% PFA (100 μL/well) for 15 minutes at room temperature.
3. The well was washed twice with 200 μL/well phosphate buffered saline (PBS).
4. 100 μL per well 0.1% Triton in PBS was added and incubated for 5 minutes, then remove.
5. The well was washed twice with 100 μL PBS per well.
6. 100 μL of Phalloidin/Hoechst/BSA solution was added and incubated at room temperature in the dark for 20 minutes.
7. The well was washed twice with 100 μL PBS.
8. The hybrid composition was image on InCell 2000.

### Example 3: Injection of a Hybrid Composition into the Heart

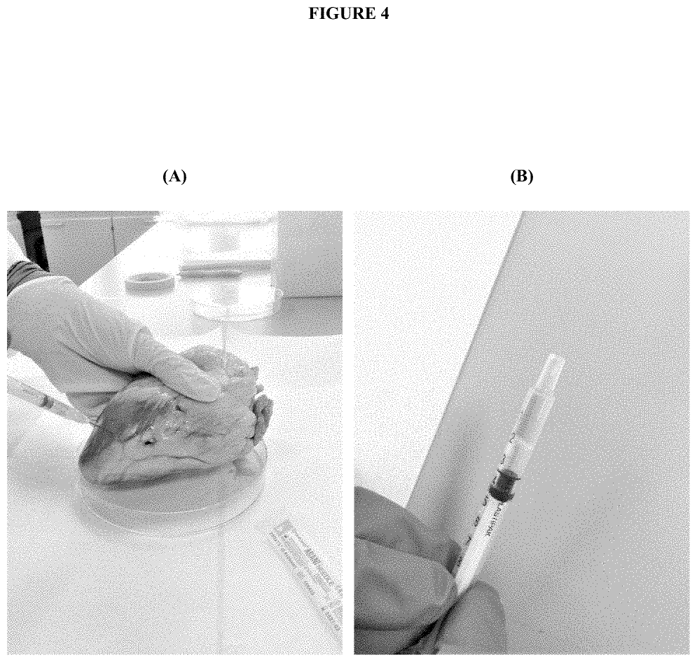

Fresh hybrid compositions were prepared by Cell Therapy Limited as described above in Example 2. 6 hours after combining the cells and scaffold, the cells had completely attached to the scaffold. The hybrid composition was removed from the well using a 20-gauge needle. The hybrid composition and medium were then injected into the left ventricle of a dead sheep's heart. This is shown in FIG. 4.

What is claimed is:

1. An elongate scaffold comprising:
an inner portion comprising a polymer; and
an outer portion comprising a porous, non-woven network of polymer fibers, wherein the packing density of the inner portion is greater than the packing density of the outer portion;
wherein the inner portion (a) comprises a plurality of polymer fibers twisted around one another or (b) is a solid core comprising the polymer.

2. A scaffold according to claim 1 wherein the outer portion is disposed around at least part of the inner portion.

3. A scaffold according to any one of claims 1 to 2 which is cylinder-shaped.

4. A scaffold according to any one of the preceding claims wherein the length of the scaffold is at least about 2 times the diameter of the scaffold, optionally at least about 5 times the diameter of the scaffold, or from about 5 to about 20000 times the diameter of the scaffold.

5. A scaffold according to any one of the preceding claims which has a length of from about 2 mm to about 4000 mm, optionally from about 4 mm to about 100 mm, or from about 4 mm to about 50 mm.

6. A scaffold according to any one of the preceding claims which has a diameter of from about 100 μm to about 1000 μm, optionally from about 300 μm to about 400 μm.

7. A scaffold according to any one of the preceding claims wherein the inner portion comprises a plurality of polymer fibers twisted around one another wherein the plurality of polymer fibers has an average number of twists, per mm of length of the inner portion, of from 0.1 to 4, optionally wherein the plurality of polymer fibers has an average number of twists, per mm of length of the inner portion, of about 1.

8. A scaffold according to any one of the preceding claims wherein the inner portion comprises at least 50 polymer fibers twisted around one another.

9. A scaffold according to any one of claims 1, 7 and 8 wherein the mean diameter of the polymer fibers in the inner and the outer portions is from about 500 nm to about 10 μm, optionally wherein the mean diameter of the polymer fibers in the inner and the outer portions is from about 1 μm to about 5 μm, or from about 2 μm to about 5 μm.

10. A scaffold according to any one of the preceding claims wherein the porous, non-woven network of polymer fibers in the outer portion has a porosity which is equal to or greater than 50%.

11. A scaffold according to any one of the preceding claims wherein the polymer in the inner portion and the polymer in the outer portion are the same or different polymers, and are polymers which are bioabsorbable and biocompatible;
optionally wherein the polymer in the inner portion, the polymer in the outer portion, or both, comprise poly(lactide), poly(glycolide), poly(lactide-co-glycolide) (PLGA) or polycaprolactone (PCL).

12. A scaffold according to any one of the preceding claims which is cylinder-shaped, wherein the cylinder-shaped scaffold comprises:
(i) said inner portion, wherein the inner portion runs the length of the scaffold and comprises aligned polymer fibers twisted around one another; and
(ii) said outer portion, wherein the outer portion is disposed around the inner portion and comprises a porous, non-woven network of polymer fibers;
wherein:
the scaffold has a length of from 4 mm to 8 mm and a diameter of from 200 μm to 500 μm; and
the polymer fibers in the inner and outer portions are fibers of poly(lactide-co-glycolide) (PLGA), poly(lactide), poly(glycolide) or polycaprolactone (PCL) having a mean diameter of from about 2 μm to about 5 μm.

13. A scaffold precursor which is an elongate strip comprising a plurality of layers of polymer fibers, the plurality of layers comprising:
a first region, comprising at least one first layer of non-woven polymer fibers;
a second region, disposed on the first region, the second region comprising at least one layer comprising aligned polymer fibers which are orientated along the length of the strip;
optionally, a third region which is disposed on the second region, which third region comprises at least one further layer of non-woven polymer fibers.

14. A scaffold precursor according to claim 13 wherein the plurality of layers has a thickness (depth) of from about 30 μm to about 1000 μm, optionally wherein the plurality of layers has a thickness (depth) of from about 80 μm to about 120 μm.

15. A scaffold precursor according to claim 13 or claim 14 wherein:
the plurality of layers has a width of from about 1 to about 15 times the thickness (depth) of the scaffold precursor, optionally from about 1 to about 4 times the thickness (depth) of the scaffold precursor; and/or
the plurality of layers has a length of at least 30 times the thickness (depth) of the scaffold precursor, optionally at least 100 times the thickness (depth) of the scaffold precursor, or at least 1000 times the thickness (depth) of the scaffold precursor.

16. A scaffold precursor according to any one of claims 13 to 15 wherein the plurality of layers comprises:
a first layer comprising non-woven polymer fibers;
a second layer, disposed on the first layer, the second layer comprising aligned polymer fibers orientated along the length of the strip; and
optionally, a third layer, disposed on the second layer, the third layer comprising non-woven polymer fibers;
wherein the plurality of layers has a thickness (depth) of from about 30 μm to about 1000 μm, a width of from about 60 μm to about 2 mm, and a length of at least 1 mm; and
the polymer fibers in the first, second and third layers are fibers of poly(lactide-co-glycolide) (PLGA), poly(lactide), poly(glycolide) or polycaprolactone (PCL) having a mean diameter of from 500 nm to 10 μm, optionally wherein the mean diameter of the polymer fibers in the first, second and third layers is from 1 μm to 5 μm.

17. A process for producing a scaffold, which process comprises twisting a scaffold precursor as defined in any one of claims 13 to 16 along its length.

18. A process according to claim 17 which comprises (i) treating a scaffold precursor as defined in any one of claims 13 to 16 with a solvent, (ii) twisting the treated scaffold precursor along its length, and (iii) optionally, annealing the scaffold.

19. A process according to claim 17 or claim 18 which further comprises cutting the twisted scaffold precursor to a particular length, optionally wherein the particular length is at least about 2 times the diameter of the twisted scaffold precursor and may be as further defined in claim 4 or claim 5.

20. A scaffold which is obtainable by a process as defined in any one of claims 17 to 19, optionally wherein the scaffold is as further defined in any one of claims 1 to 12.

21. A hybrid composition comprising:
(i) cells, a drug, a nucleic acid, a nucleotide, a protein, a polypeptide or an exosome, optionally wherein the nucleic acid comprises DNA, RNA, RNAi, SaRNA or SiRNA; and
(ii) a scaffold as defined in any one of claims 1 to 12 and 20;
optionally wherein the hybrid composition comprises cells and said scaffold, and the cells are attached to the surface of the scaffold, are disposed in pores of the scaffold, or both;
preferably wherein the cells are therapeutic cells, even more preferably adherent therapeutic cells.

22. A hybrid composition according to claim 21 which comprises cells and said scaffold, wherein the outer portion of the scaffold comprises the cells.

23. A process for producing a hybrid composition as defined in claim 22 or 22, comprising combining (i) a scaffold as defined in any one of claims 1 to 12 and 20 and (ii) cells, a drug, a nucleic acid, a nucleotide, a protein, a polypeptide or an exosome in a culture vessel.

24. A process for producing a hybrid composition as defined in claim 21 or 22, comprising: (i) combining a scaffold as defined in any one of claims 1 to 12 and 20 which is cylinder-shaped, and adherent therapeutic cells, in a culture vessel which is not modified to facilitate the attachment of cells; and (ii) allowing the cells to infiltrate and proliferate on the surface and within the outer portion of the scaffold and thereby producing said hybrid composition.

25. A process for producing a hybrid composition as defined in claim 21 or 22, comprising (i) combining a scaffold as defined in any one of claims 1 to 12 and 20 which is cylinder-shaped, and adherent therapeutic cells, in a culture vessel which is not modified to facilitate the attachment of cells, wherein the adherent therapeutic cells comprise stem cells, for example progenitor cells of mesodermal lineage (PMLs), immuno-modulatory progenitor (iMP) cells, immuno-oncology mesodermal progenitor cells (ioMP) cells or a combination thereof; and (ii) allowing the adherent therapeutic cells to attach to the scaffold and thereby producing said hybrid composition.

26. A method of repairing a damaged or diseased tissue in a patient, comprising contacting the damaged or diseased tissue with one or more hybrid compositions as defined in claim 21 or 22 and thereby treating the damaged or diseased tissue in the patient, optionally wherein the hybrid composition comprises adherent therapeutic cells and said scaffold and the composition comprises a therapeutically effective number of the adherent therapeutic cells.

27. A method according to claim 26, wherein the method comprises implanting or injecting the hybrid composition into the damaged or diseased tissue or adhering or anchoring the hybrid composition to the damaged or diseased tissue.

28. A hybrid composition as defined in claim 21 or 22 for use in a method for treatment of the human or animal body by therapy.

29. A hybrid composition as defined in claim 21 or 22 for use in a method of treating a cardiac, bone, cartilage, tendon, ligament, liver, kidney, joint, spleen, eye, spinal disc, connective tissue, or lung injury or disease in a patient, or for use as a tissue filler in a reconstructive or cosmetic procedure, or for use in wound healing.
